(19) United States
(12) Patent Application Publication
(10) Pub. No.: US 2026/0022191 A1
(43) Pub. Date: Jan. 22, 2026

(54) METHOD OF PREPARING AN ANTIBODY WITH THIOL GROUP SITE-SPECIFIC MODIFICATIONS AND USE OF TCEP

(71) Applicant: SUZHOU BIOREINNO BIOTECHNOLOGY LIMITED COMPANY, Suzhou, Jiangsu (CN)

(72) Inventors: Lili WU, Suzhou City, Jiangsu Province (CN); Ao JI, Suzhou, Jiangsu (CN); Yaru SHAO, Suzhou, Jiangsu (CN)

(21) Appl. No.: 18/993,525

(22) PCT Filed: Aug. 22, 2023
(86) PCT No.: PCT/CN2023/114317

§ 371 Date: Jan. 10, 2025

(30) Foreign Application Priority Data
Aug. 22, 2022 (WO) PCT/CN2022/113992
Sep. 20, 2022 (WO) PCT/CN2022/119955
Jan. 19, 2023 (WO) PCT/CN2023/073070

(51) Int. Cl. C07K 16/32; A61K 47/68; C07K 16/28; C07K 16/30

(52) U.S. Cl. CPC C07K 16/32; A61K 47/68031; C07K 16/2878; C07K 16/30; C07K 2317/24

(57) ABSTRACT

Provided is a method of preparing antibody with thiol group site-specific modifications and use of TCEP. The thiol group(s) is/are reduced from the interchain disulfide bonds within the antibody, and the method comprises using TCEP or a salt thereof and transition metal ions, wherein, the molar ratio of TCEP and the transition metal ions is 1:0.4 to 1:200. The antibody with thiol group site-specific modification is the ADC with D1, the ADC with D2, the ADC with D1+D6, the ADC with D1+D3, the ADC with D2+D6, the ADC with D2+D3, the ADC with D0+D6, or the ADC with D0+D3, As compared with conventional conjugation method, the homogeneity of ADCs produced from the method can be dramatically improved. Specifically, the content of the ADC with D2 in general more than 55%, 60%, 65%, even to 70%, 75%, 80% or 83%.

### CROSS-REFERENCE TO RELATED APPLICATIONS

This application claims the priority to PCT Application No. PCT/CN2022/113992, filed on Aug. 22, 2022, PCT Application No. PCT/CN2022/119955, filed on Sep. 20, 2022, and PCT Application No. PCT/CN2023/073070, filed on Jan. 19, 2023. The contents of the prior PCT applications are considered as a part of the present disclosure and are incorporated herein in its entirety.

### TECHNICAL FIELD

The present application relates to a method of preparing an antibody with thiol group site-specific modifications and use of TCEP or a salt thereof. Specifically, the present application relates to a bio-conjugation process for preparing ADCs with improved homogeneity.

### BACKGROUND

The statements in this section merely provide background information related to the present disclosure and do not necessarily constitute prior art.

Antibody drug conjugates (ADCs) are antibodies with thiol group modifications in which an antibody is linked to small molecule drugs with linkers. ADCs ideally combine the specificity of antibodies and high potency of cytotoxic drugs by delivering potent cytotoxic drugs to antigen-expressing cells, thereby enhancing their targeted cytotoxic activity. In contrast to traditional chemotherapeutic drugs, ADCs target only antigen-expressing cancer cells so that healthy cells are less severely affected (Pettinato, Mark C. (2021) “Introduction to Antibody-Drug Conjugates.” Antibodies (Basel, Switzerland) 10(42): 42-52, Joubert N, Beck A, Dumontet C, Denevault-Sabourin C. (2020) “Antibody-Drug Conjugates: The Last Decade.” Pharmaceuticals (Basel). 13(9): 245-275). ADCs have extensive potential therapeutic applications in several disease areas, especially in cancer, and become a novel targeted drug for disease treatment. Since the approvals of Mylotarg in 2000, so far fourteen ADC drugs have been approved by US Food and Drug Administration.

For drug attachment of ADCs, functional groups with high reactivity on both antibody and linker-payload (i.e., linker-drug) were used for the conjugation, to form stable covalent bonds. Conventional means of conjugation, i.e., covalent bonding of a drug moiety to an antibody via a linker, generally leads to a heterogeneous mixture of molecules where the drug moieties are attached at several sites on the antibody. For example, ADCs are usually produced by two conventional chemical strategies, lysine-based conjugation and cysteine from the reduction of interchain disulfide bond based conjugation. For cysteine from the reduction of interchain disulfide bond based conjugation, it comprises a step of reducing interchain disulfide bonds in the presence of various reductants, followed by nucleophilic reaction of thiol groups. In this conjugation process, ADCs are typically formed by conjugating one or more antibody cysteine thiol groups to one or more linker-payload moieties thereby generating a heterogeneous antibody drug conjugate mixture (for example, Adcetris) where the drug moieties are attached at several sites on the antibody. For an ADC with a Drug-Antibody-Ratio (DAR) around 4, the heterogeneous mixture typically contains a distribution of antibodies attached with drug moieties from 0 to about 8, or more. In addition, within each subgroup of conjugates with a particular integer ratio of drug moieties to a single antibody, there is a potentially heterogeneous mixture where the drug moiety is attached at various sites on the antibody. The heterogeneous mixture is so complex that each conjugation product potentially has different pharmacokinetic, toxicity and efficacy profiles. Meanwhile it is difficult and expensive to characterize and purify them. And the conventional non-specific conjugation and conjugate distribution are largely influenced by factors such as pH, temperature, concentration, salt concentration, and co-solvents, so establishing a robust conjugation process always is challenging.

A number of methods have been developed to improve the homogeneity of ADCs. For example, Synaffix's technology GlycoConnect™ (US2015/0320882, synaffix.com/platform/technology/) has been developed to covert an antibody into a stably conjugated ADC with DAR2, DAR4 or even DAR1 and DAR6, by modifying the native antibody glycan through a three-step process: enzyme digestion, enzyme mediated ligation and metal-free click chemistry. However, this technology suffers from several disadvantages, including but not limited to high cost, complicated operation, and loss of Fc function.

US20210128743 discloses a method for generating an ADC by means of a microbial transglutaminase (MTG). The method comprises a step of conjugating a linker having a primary amine residue, to a Gln residue (in most cases, N295 in an IgG1 antibody) comprised in the heavy or light chain of an antibody. Besides the disadvantages associated with the use of enzyme catalysis, due to proximity of N295 to N297, the modification would strongly affect Fc function, such as ADCC, ADCP and CDC. (Jeger S, Zimmermann K, Blanc A, et al. Site-specific and stoichiometric modification of antibodies by bacterial transglutaminase. Angew Chem Int Ed Engl. 2010 Dec. 17; 49(51):9995-7.) Moreover, due to the use of ligase, at least one-step chromatography purification is necessary.

Both pClick (US20210130395) and AbYlink (WO2021/110860) take advantage of Fc affinity peptide to install lysine reactive linker-payloads through proximity promoted ligation, generating antibody conjugates with DAR 2. In contrast to thiol modification, lysine modification consumes surface charge residues of the antibody, and so may alter intrinsic conformation and affect stability of the antibody. Besides, the conjugation sites directed by Fc affinity peptide are close or partially overlapping with Fc receptor binding domains, so would cause at least partial loss of Fc function.

Therefore, there is a continuing need for developing a novel bio-conjugation process which can generate ADCs with improved homogeneity.

### SUMMARY

The present application develops a method of preparing an antibody with thiol group site-specific modifications and use of TCEP or a salt thereof. With the thiol group site-specific modifications of the antibody, the present application provides many kinds of ADCs with high homogeneity, such as the ADC with D2, the ADC with D1, the ADC with D2+D6, the ADC with D2+D3, the ADC with D1+D6, the ADC with D1+D3, the ADC with D0+D6, the ADC with D0+D3, the ADC with D2+D2, the ADC with D2+D4, the ADC with D1+D4 or the ADC with D1+D2. As compared with conventional conjugation process, the homogeneity of ADC with D2 is up to 55%, 60%, 65%, even to 70%, 75%, 80% or 83%. Meanwhile the method has simple manipulation and reduced cost without enzymes engineering and glycan modification. The ADCs with improved homogeneity generated by the method of the present application further have optimized safety and efficacy.

On the one aspect, the present application provides a method of preparing an antibody with thiol group site-specific modifications, which characterized in that, the thiol group(s) is/are reduced from the interchain disulfide bonds within the antibody, and the method comprises using tris (2-carboxyethyl) phosphine (TCEP) or a salt thereof and transition metal ions, wherein, the molar ratio of TCEP and the transition metal ions is 1:0.4 to 1:200.

On the second aspect, the present application provides an antibody with thiol group site-specific modifications prepared by the method of the present application.

On the third aspect, the present application provides a pharmaceutical composition comprising the antibody with thiol group site-specific modifications according to the present application and one or more of pharmaceutically acceptable carrier.

On the fourth aspect, the present application provides use of TCEP or the salt thereof in the preparation of the antibody with thiol group site-specific modifications according to the present application.

On the fifth aspect, the present application provides use of the antibody with thiol group site-specific modifications according to the present application in the manufacture of a therapeutic agent for diagnosing, preventing or treating a disease.

On the sixth aspect, the present application provides a method of preventing or treating a disease in a subject in need thereof, comprising administrating to the subject a therapeutically effective amount of the antibody with thiol group site-specific modifications according to the present application.

In the present application, TCEP and the transition metal ions with specific molar ratio selectively reduce one of four interchain disulfide bonds within antibody. In step (b), the modification reagent 1 is attached to the reduced thiol groups of the antibody. Without introducing the transition metal ions in step (c), the second reductant is introduced to reduce the other interchain disulfide bonds within the antibody. With introducing the transition metal ions in step (c), the second reductant is introduced to reduced one or two of the remaining interchain disulfide bond within the antibody. In step (d), the modification reagent 2 is introduced to modify the reduced thiol groups from step (c). By means of the above, the present application provides many kinds of ADC with high homogeneity without enzymes engineering and glycan modification. For example, the homogeneity of the ADC with D2 is up to 55%, 60%, 65%, even to 70%, 75%, 80% or 83%. The homogeneity of the ADC with D1 is up to 70%, 75%, even to 77% or 80%. The homogeneity of the ADC with D0+D6 is up to 65%, 70%, even to 73% or 75%. The homogeneity of the ADC with D2-D6 is up to 65%, 70%, 72%, even to 75%. The method of the present application is compatible with current thiol-reactive linker-drug technologies and provides a high content of ADCs with minimum conformation change and intact Fc function. Meanwhile it has simple manipulation and reduced cost.

### BRIEF DESCRIPTION OF THE DRAWINGS

FIG. 1 shows HIC-HPLC (Hydrophobic interaction chromatography-High performance liquid chromatography) of Trastuzumab-[MC-VC-PAB-MMAE]₂ conjugate prepared by using TCEP and the transition metal ions of example 1.

FIG. 2 shows HIC-HPLC of Sacituzumab-[MC-VC-PAB-MMAE]₂ conjugate prepared by using TCEP and the transition metal ions of example 2.

FIG. 3 shows HIC-HPLC of Belantamab-[MC-VC-PAB-MMAE]₂ conjugate prepared by using TCEP and the transition metal ions of example 3.

FIG. 4-13 show HIC-HPLC of Transtuzumab-[MC-VC-PAB-MMAE]₂ conjugate of example 4-13 (the molar ratio of TCEP and Zn²⁺ is 1:0.4, 1:1, 1:2, 1:4, 1:6, 1:8, 1:10, 1:12, 1:14, 1:16).

FIG. 14A-D show the HIC-HPLC of Transtuzumab-[MC-VC-PAB-MMAE]₂ conjugate of examples 14-16 and the comparative example 4 (the molar ratio of TCEP and Zn²⁺ is 1:30, 1:70, 1:125, 1:250).

FIG. 15A-D show the HIC-HPLC of Transtuzumab-[MC-VC-PAB-MMAE]₂ conjugate of examples 17-20 (the molar ratio of TCEP and the antibody is 1.2:1, 2.0:1, 2.5:1, 3.0:1).

FIG. 16 shows HIC-HPLC of Transtuzumab-[MC-VC-PAB-MMAE]₂ conjugate prepared by using the MOPS buffer (the pH value is 6.7) of example 21.

FIG. 17 shows HIC-HPLC of Transtuzumab-[MC-VC-PAB-MMAE]₂ conjugate prepared by using the MOPS buffer (the pH value is 7.0) of example 22.

FIG. 18 shows HIC-HPLC of Transtuzumab-[MC-VC-PAB-MMAE]₂ conjugate prepared by using the MOPS buffer (the pH value is 7.4) of example 23.

FIG. 19 shows HIC-HPLC of Transtuzumab-[MC-VC-PAB-MMAE]₂ conjugate prepared by using the Bis-Tris buffer (the pH value is 6.7) of example 24.

FIG. 20 shows HIC-HPLC of Transtuzumab-[MC-VC-PAB-MMAE]₂ conjugate prepared by using the PIPES buffer (the pH value is 6.7) of example 25.

FIG. 21 shows HIC-HPLC of Transtuzumab-[MC-VC-PAB-MMAE]₂ conjugate prepared by using the BES buffer (the pH value is 6.7) of example 26.

FIG. 22 shows HIC-HPLC of Transtuzumab-[MC-VC-PAB-MMAE]₂ conjugate prepared by using the MES buffer (the pH value is 6.7) of example 27.

FIG. 23 shows HIC-HPLC of Transtuzumab-[MC-VC-PAB-MMAE]₂ conjugate prepared by using the HEPES buffer (the pH value is 6.7) of example 28.

FIG. 24 shows HIC-HPLC of Transtuzumab-[MC-VC-PAB-MMAE]₂ conjugate prepared by using the DIPSO buffer (the pH value is 7.4) of example 29.

FIG. 25 shows HIC-HPLC of Transtuzumab-[MC-VC-PAB-MMAE]₂ conjugate prepared by using the MOPSO buffer (the pH value is 7.4) of example 30.

FIG. 26 shows HIC-HPLC of Transtuzumab-[MC-VC-PAB-MMAE]₂ conjugate prepared by using the TES buffer (the pH value is 7.4) of example 31.

FIG. 27 shows HIC-HPLC of Transtuzumab-[MC-VC-PAB-MMAE]₂ conjugate prepared by using the ACES buffer (the pH value is 7.4) of example 32.

FIG. 28A-B show HIC-HPLC of Transtuzumab-[MC-VC-PAB-MMAE]₂ conjugate prepared by using the MES buffer (the pH value is 5.8, 6.4) of example 33-34.

FIG. 29A-C show HIC-HPLC of Transtuzumab-[MC-VC-PAB-MMAE]₂ conjugate prepared with different reduction temperature in step (1) of example 35-37.

FIG. 30A-G show HIC-HPLC of Transtuzumab-[MC-VC-PAB-MMAE]₂ conjugate prepared with different incubation time in step (1) of example 38-44.

FIG. 31 shows HIC-HPLC of Trastuzumab-[Maleimide-PEG4-N3-DBCO-Cy3]₁ conjugate of example 45.

FIG. 32A shows HIC-HPLC of Trastuzumab-[Maleimide]₁ of example 46; B shows HIC-HPLC of Trastuzumab-[Maleimide]: [MC-VC-PAB-MMAE]₆ conjugate of example 46.

FIG. 33A shows HIC-HPLC of Trastuzumab-[MC-VC-PAB-MMAE]₂ of example 47; B shows HIC-HPLC of Trastuzumab-[MC-VC-PAB-MMAE]₂[MC-GGFG-DXd]₆ conjugate of example 47.

FIG. 34. HIC-HPLC of Transtuzumab-[MC-VC-PAB-MMAE]₂ conjugate prepared without the transition metal ions of comparative example 1.

FIG. 35. HIC-HPLC of Sacituzumab-[MC-VC-PAB-MMAE]₂ conjugate prepared without the transition metal ions of comparative example 2.

FIG. 36. HIC-HPLC of Belantamab-[MC-VC-PAB-MMAE]₂ conjugate prepared without the transition metal ions of comparative example 3.

FIG. 37. HIC-HPLC of Transtuzumab-[MC-VC-PAB-MMAE]₂ conjugate prepared by using the PB (the pH value is 5.8) of comparative example 5.

FIG. 38. HIC-HPLC of Transtuzumab-[MC-VC-PAB-MMAE]₂ conjugate prepared by using the PB (the pH value is 6.2) of comparative example 6.

FIG. 39. HIC-HPLC of Transtuzumab-[MC-VC-PAB-MMAE]₂ conjugate prepared by using the PB (the pH value is 6.7) of comparative example 7.

FIG. 40. HIC-HPLC of Transtuzumab-[MC-VC-PAB-MMAE]₂ conjugate prepared by using the PB (the pH value is 7.0) of comparative example 8.

FIG. 41. HIC-HPLC of Transtuzumab-[MC-VC-PAB-MMAE]₂ conjugate prepared by using the PB (the pH value is 7.4) of comparative example 9.

FIG. 42. HIC-HPLC of Transtuzumab-[MC-VC-PAB-MMAE]₂ conjugate prepared by using the ADA buffer (the pH value is 6.7) of comparative example 10.

FIG. 43. HIC-HPLC of Transtuzumab-[MC-VC-PAB-MMAE]₂ conjugate prepared by using the MOBS buffer (the pH value is 7.4) of comparative example 11.

FIG. 44. HIC-HPLC of Transtuzumab-[MC-VC-PAB-MMAE]₂ conjugate prepared by using the TAPSO buffer (the pH value is 7.4) of comparative example 12.

### DETAILED DESCRIPTION

The present disclosure is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following description is intended to illustrate some particular embodiments of the invention, and not to exhaustively specify all permutations, combinations and variations thereof.

Unless defined otherwise, all technical and scientific terms used herein have the same meaning as commonly understood by one of ordinary skill in the art to which the disclosure pertains. Although any methods and materials similar or equivalent to those described herein may be used in the practice for testing of the present disclosure, the preferred materials and methods are described herein. In describing and claiming the present disclosure, the following terminology will be used.

In order to better understand the disclosure, the definitions and explanations of the relevant terms are provided as follows.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “an antibody” includes a plurality of antibodies; reference to “a transition metal ion” includes mixtures of transition metal ions, and the like. In this application, the use of “or” means “and/or” unless stated otherwise.

Throughout this disclosure, unless the context requires otherwise, the words “comprise”, “comprises”, “comprising”, “contain”, “contains” and “containing” will be understood to imply the inclusion of a stated step or element or group of steps or elements but not the exclusion of any other step or element or group of steps or elements. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “about” or “approximately” refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

As used herein, the term “the ADC with D2” or “D2” refers to the ADC in which two drug molecules are coupled to one single antibody molecule, where two drug molecules may be coupled to —SH groups generated by reduction of S—S bonds between heavy and light chains via linkers, or may be coupled to —SH groups generated by reduction of S—S bonds between heavy and heavy chains via linkers.

As used herein, the term “the ADC with D0” or “D0” refers to the ADC in which the number of drugs coupling to a single antibody molecule is zero.

As used herein, the term “the ADC with D1” or “D1” refers to the ADC in which one of the thiobridge group bearing the linker-payload re-bridges two thiol groups of one single antibody molecule.

As used herein, the term “the ADC with D4” or “D4” refers to the ADC in which four drug molecules are coupled to one single antibody molecule, where four drug molecules may be coupled to four —SH groups generated by reduction of two S—S bonds between heavy and light chains via linkers, or four drug molecules may be coupled to four —SH groups generated by reduction of two S—S bonds between heavy and heavy chains via linkers, or two drug molecules may be coupled to two —SH groups generated by reduction of one S—S bond between heavy and light chains via linkers and the other two drug molecules may be coupled to two —SH groups generated by reduction of one S—S bond between heavy and heavy chains vis linkers.

As used herein, the term “the ADC with D6” or “D6” refers to the ADC in which six drug molecules are coupled to one single antibody molecule, where six drug molecules may be coupled to six —SH groups generated by reduction of three S—S bonds.

As used herein, the term “the ADC with D8” or “D8” refers to the ADC in which eight drug molecules are coupled to one single antibody molecule, where eight drug molecules may be coupled to eight —SH groups generated by reduction of four S—S bonds.

As used herein, the term “the ADC with D1+D6” or “D1+D6” refers to the ADC in which one of the first thiobridge group bearing the first linker-payload re-bridging two thiol groups and six of the second linker-payloads are coupled to one single antibody molecule, wherein, the first linker-payload and the second linker-payload may be same or different.

As used herein, the term “the ADC with D1+D3” or “D1+D3” refers to the ADC in which one of the first thiobridge group bearing the first linker-payload and three of the second thiobridge groups bearing the second linker-payload re-bridge eight thiol groups of one single antibody molecule, wherein, the first thiobridge group and the second thiobridge group may be same or different, and the first linker-payload and the second linker-payload may be same or different.

As used herein, the term “the ADC with D2+D6” or “D2+D6” refers to the ADC in which two of the first linker-payloads and six of the second linker-payloads are coupled to one single antibody molecule, wherein, the first linker-payload and the second linker-payload may be same or different.

As used herein, the term “the ADC with D2+D3” or “D2+D3” refers to the ADC in which two of the first linker-payloads are coupled to one single antibody and three of the second thiobridge groups bearing the second linker-payload re-bridging six thiol groups of the single antibody molecule, wherein, the first linker-payload and the second linker-payload may be same or different.

As used herein, the term “the ADC with D0+D6” or “D0+D6” refers to the ADC in which one of the first thiobridge group re-bridging two thiol groups and six of the second linker-payloads are coupled to one single antibody molecule, or refers to the ADC in which two of the end capping reagents and six of the second linker-payloads are coupled to one single antibody molecule.

As used herein, the term “the ADC with D0+D3” or “D0+D3” refers to the ADC in which one of the first thiobridge group re-bridges two thiol groups and three of the second thiobridge group bearing the second linker-payload re-bridge six thiol groups of one single antibody molecule, wherein, the first thiobridge group and the second thiobridge group may be same or different. In some embodiments, D0+D3 refers to the ADC in which two of the end capping reagents react with two thiol groups and three of the second thiobridge group bearing the linker-payload re-bridge six thiol groups of the single antibody molecule.

As used herein, the term “the ADC with D2+D4” or “D2+D4” refers to the ADC in which two of the first linker-payloads and four of the second linker-payloads are coupled to one single antibody molecule.

As used herein, the term “the ADC with D4-D2” or “D4+D2” refers to the ADC in which four of the first linker-payloads and two of the second linker-payloads are coupled to one single antibody molecule.

As used herein, the term “the ADC with D4+D4” or “D4-D4” refers to the ADC in which four of the first linker-payloads and four of the second linker-payloads are coupled to one single antibody molecule.

As used herein, the term “HEPES buffer” refers to 4-hydroxyethyl piperazine ethanesulfonic acid buffer.

As used herein, the term “PBS” refers to phosphate buffer saline.

As used herein, the term “PB’ refers to phosphate buffer.

As used herein, the term “MES buffer” refers to 2-(N-morpholino) ethanesulfonic acid buffer.

As used herein, the term “BES buffer” refers to N,N-Bis (2-hydroxyethyl)-2-aminoethanesulphonic acid buffer.

As used herein, the term “MOPS buffer” refers to 3-morpholinopropanesulfonic Acid buffer.

As used herein, the term “Bis-Tris buffer” refers to Bis(2-hydroxyethyl)amino-tris(hydroxymethyl)methane buffer.

As used herein, the term “PIPES buffer” refers to piperazine-1,4-bisethanesulfonic acid buffer.

As used herein, the term “DIPSO buffer” refers to 3-[bis(2-hydroxyethyl)amino]-2-hydroxypropanesulphonic acid buffer.

As used herein, the term “MOBS buffer” refers to 4-(N-morpholino)butanesulfonic Acid buffer.

As used herein, the term “MOPSO buffer” refers to 3-(N-morpholino)-2-hydroxy-1-propanesulfonic acid buffer.

As used herein, the term “TES buffer” refers to 2-[tris(hydroxymethyl)methylamino]-1-ethanesulfonic acid buffer.

As used herein, the term “ACES buffer” refers to N-(carbamoylmethyl)taurine buffer.

As used herein, the term “TAPSO buffer” refers to 3-[N-tris-(hydroxymethyl)methylamino]-2-hydroxypropanesulphonic acid buffer.

As used herein, the term “ADA buffer” refers to N-(Carbamoylmethyl) iminodiacetic acid buffer.

As used herein, the term “BTP buffer” refers to Bis-tris propane buffer.

As used herein, the term “Heppso buffer” refers to N-(Hydroxyethyl) piperazine-N′-2-hydroxypropanesulfonic acid buffer.

As used herein, the term “POPSO buffer” refers to piperazine-N,N′-bis(2-hydroxy-propane sulfonic) acid buffer.

As used herein, the term “EPPS buffer” refers to 4-(2-Hydroxyethyl)-1-piperazinepropanesulfonic acid buffer.

As used herein, the term “Tris buffer” refers to tris(hydroxymethyl)aminomethane buffer.

As used herein, the term “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “Antibody drug conjugate” or “Antibody drug conjugates” or “ADC” or “the ADC” or “the ADCs” or “ADCs” or “an ADC” refers to a conjugate formed by covalently coupling drugs to an antibody directly or indirectly via one or more suitable linkers. ADC is generally in a format of antibody-linker-drug conjugate. The ADCs combine ideal properties of both antibodies and cytotoxic drugs by targeting potent cytotoxic drugs to the antigen-expressing tumor cells, thereby enhancing their anti-tumor activity.

As used herein, the term “payload” refers to any cytotoxic molecule or any molecule of medical interest bears at least one substituted group or a partial structure allowing connection to a linker structure. The payload may kill cancer cells and/or inhibit growth, proliferation, or metastasis of cancer cells, thereby reducing, alleviating, or eliminating one or more symptoms of a disease or disorder.

As used herein, the term “linker” refers to a substituted molecule which contains at least two substituted groups, one of which can covalently bond a drug molecule and the other of which can covalently couple to an antibody or the reactive groups of the thiobridge reagent.

As used herein, the term “antibody” refers to any immunoglobulin, monoclonal antibody, polyclonal antibody, multispecific antibody, or bispecific (bivalent) antibody that binds to a specific antigen. A native intact antibody comprises two heavy chains and two light chains. Each heavy chain consists of a variable region (“HCVR”) and a first, second, and third constant region (CH1, CH2 and CH3), while each light chain consists of a variable region (“LCVR”) and a constant region (CL). Mammalian heavy chains are classified as α, δ, ε, γ and μ, and mammalian light chains are classified as λ or κ. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light (L) chain CDRs including LCDR1, LCDR2, and LCDR3, heavy (H) chain CDRs including HCDR1, HCDR2, HCDR3). CDR boundaries for antibodies may be defined or identified by the conventions of Kabat, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3): 651-63 (1985); Chothia, C. and Lesk, A. M., J. Mol. Biol., 196, 901 (1987); Chothia, C. et al., Nature. December 21-28; 342(6252): 877-83 (1989); Kabat E. A. et al., National Institutes of Health, Bethesda, Md. (1991)). The three CDRs are interposed between flanking stretches known as framework regions (FRs), which are more highly conserved than the CDRs and form a scaffold to support the hypervariable loops. Each HCVR and LCVR comprises four FRs, and the CDRs and FRs are arranged from amino terminus to carboxy terminus in the order: FR1, CDR1, FR2, CDR2, FR3, CDR3, FR4. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

As used herein, the term “variable domain” with respect to an antibody as used herein refers to an antibody variable region or a fragment thereof comprising one or more CDRs. Although a variable domain may comprise an intact variable region (such as HCVR or LCVR), it is also possible to comprise less than an intact variable region yet and still retain the capability of binding to an antigen or forming an antigen-binding site.

As used herein, the term “antigen-binding moiety” refers to an antibody fragment formed from a portion of an antibody comprising one or more CDRs, or any other antibody fragment that binds to an antigen but does not comprise an intact native antibody structure. Examples of antigen-binding moiety include, without limitation, a variable domain, a variable region, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a multispecific antibody, a camelized single domain antibody, a nanobody, a domain antibody, and a bivalent domain antibody. An antigen-binding moiety is capable of binding to the same antigen to which the parent antibody binds. In certain embodiments, an antigen-binding moiety may comprise one or more CDRs from a particular human antibody grafted to a framework region from one or more different human antibodies. For more and detailed formats of antigen-binding moiety are described in Spiess et al, 2015 (Supra), and Brinkman et al., mAbs, 9(2), pp. 182-212 (2017), which are incorporated herein by their entirety.

As used herein, the term “Fab” with regard to an antibody refers to that portion of the antibody consisting of a single light chain (both variable and constant regions) associating to the variable region and first constant region of a single heavy chain by a disulfide bond.

As used herein, the term “F(ab′)2” refers to a dimer of Fab′.

As used herein, the term “Fc” with regard to an antibody refers to that portion of the antibody consisting of the second (CH2) and third (CH3) constant regions of a first heavy chain bound to the second and third constant regions of a second heavy chain via disulfide bonding. The Fc portion of the antibody is responsible for various effector functions such as ADCC, and CDC, but does not function in antigen binding.

As used herein, the term “monoclonal antibody” refers to an antibody obtained from a population of substantially homogenous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see for example: U.S. Pat. Nos. 4,816,567; 5,807,715). The monoclonal antibodies may also be isolated from phage antibody libraries using the techniques described in Clackson et al. (1991) Nature, 352:624-628; Marks et al. (1991) J. Mol. Biol., 222:581-597; for example. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape, etc.) and human constant region sequences.

As used herein, the term “chimeric” antibody refers to an antibody in which a portion of the heavy and/or light chain is derived from a particular source or species, while the remainder of the heavy and/or light chain is derived from a different source or species.

As used herein, the term “a human antibody” refers to one which possesses an amino acid sequence which corresponds to that of an antibody produced by a human or a human cell or derived from anon-human source that utilizes human antibody repertoires or other human antibody-encoding sequences. This definition of a human antibody specifically excludes a humanized antibody comprising non-human antigen-binding residues.

As used herein, the term “a humanized antibody” refers to a chimeric antibody comprising amino acid residues from non-human heavy chain variable regions (HVRs) and amino acid residues from human FRs. In certain embodiments, a humanized antibody will comprise substantially all or at least onc, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

As used herein, the term “a disulfide bond” refers to a covalent bond with the structure R—S—S—R′. The amino acid cysteine comprises a thiol group that can form a disulfide bond with a second thiol group, for example from another cysteine residue. The disulfide bond can be formed between the thiol groups of two cysteine residues residing respectively on the two polypeptide chains, thereby forming an interchain bridge or interchain bond.

As used herein, the term “transition metal”, refers to the elements of groups 4-12, justified by their typical chemistry, i.e., a large range of complex ions in various oxidation states, colored complexes, and catalytic properties either as the element or as ions (or both). Sc and Y in Group 3 are also generally recognized as transition metals.

As use herein, the term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

As use herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

As use herein, the term “subject” refers to mammals, primates (e.g., humans, male or female), dogs, rabbits, guinea pigs, pigs, rats and mice. In certain embodiments, the subject is a primate. In yet other embodiments, the subject is a human.

As use herein, the term “cancer” refers to any or a tumor or a malignant cell growth, proliferation or metastasis-mediated, solid tumors and non-solid tumors such as leukemia and initiate a medical condition. A “tumor” comprises one or more cancerous cells.

As use herein, the term “treat”, “treatment”, “treating” or “treated” of any disease refers to alleviating or ameliorating the disease (i.e., slowing or arresting the development of the disease or at least one of the clinical symptoms thereof); or alleviating or ameliorating at least one physical parameter or biomarker associated with the disease, including those which may not be discernible to the patient. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, delaying the development of a tumor, or some combination thereof.

As used herein, the term “prevent”, “preventing” or “prevention” of any disease refers to the prophylactic treatment of the disease; or delaying the onset or progression of the disease.

As used herein, the term “a therapeutically effective amount” refers to an amount of the ADC of the present application that will elicit the biological or medical response of a subject, for example, ameliorate symptoms, alleviate conditions, slow or delay disease progression, or prevent a disease, etc.

As used herein, the term “homogeneity of the ADC with Dx” refers to that the weight content of the ADC with Dx in all the ADCs produced by the method, wherein, Dx maybe D1, D2, D1+D6, D1+D3, D2+D6, D2+D3, D0+D6 or D0+D3.

Method of Preparing an Antibody with Thiol Group Site-Specific Modifications

On the one aspect, the present application provides a method of preparing an antibody with thiol group site-specific modifications, the thiol group(s) is/are reduced from the interchain disulfide bonds within the antibody, and the method comprises using tris (2-carboxyethyl) phosphine (TCEP) or a salt thereof and transition metal ions, wherein, the molar ratio of TCEP and the transition metal ions is 1:0.4 to 1:200.

In some embodiments of the present application, the number of the thiol group(s) is/are 1, 2, 3, 4, 5, 6, 7 or 8.

In some embodiments of the present application, the number of the thiol groups is 2 or 8.

In some embodiments of the present application, the interchain disulfide bonds connected the two upper heavy chains in the hinge region, or the heavy chain to the light chain in the Fab region.

In some embodiments of the present application, the interchain disulfide bonds connected the two heavy chains in the hinge region, and the heavy chain to the light chain in the Fab region.

In some embodiments of the present application, the site-specific modification dose not refer to enzyme technologies and glycan modification.

In some embodiments of the present application, the method comprises the following steps:
(a) incubating TCEP or the salt thereof and the antibody in the presence of the transition metal ions in a first buffer system to selectively reduce the interchain disulfide bonds within the antibody, which allows a bearing of two reduced thiol groups of the antibody, the molar ratio of TCEP and the transition metal ions is 1:0.4 to 1:70;
(b) introducing metal chelators and a modification reagent 1 to react with the reduced thiol groups resulted from step (a) in the first buffer system, wherein, the modification reagent 1 is an end capping reagent, a first linker-payload or a first thiobridge reagent, optionally, the first thiobridge reagent bears the first linker-payload or reactive groups.

In some embodiments of the present application, when the first thiobridge reagent bears the reactive groups, the step (b) comprises the following step:
introducing metal chelators and the first thiobridge reagent bearing reactive groups to re-bridge the reduced thiol groups resulted from step (a), then, incubating the first linker-payload in the first buffer system to react with the reactive groups of the thiobridge group.

In some embodiments of the present application, the method further comprises the following steps:
(c) incubating the reaction product from (b) and a second reductant in a second buffer system to reduce the interchain disulfide bonds in the reaction product, optionally, introducing the transition metal ions;
(d) introducing the incubation product form step (c) and a modification reagent 2 to react with the reduced thiol groups resulted from step (c), optionally, introducing the metal chelators, wherein, the modification reagent 2 is a second linker-payload or a second thiobridge reagent, optionally, the second thiobridge reagent bears the second linker-payload or reactive groups.

In some embodiments of the present application, when the second thiobridge reagent bears the reactive groups, the step (d) comprises the following steps:
introducing the incubation product from step (c) and the second thiobridge reagent bearing reactive groups to re-bridge the reduced thiol groups resulted from step (c), optionally, introducing the metal chelators, then, incubating the second linker-payload in the second buffer system to react with the reactive groups of the thiobridge group.

In some embodiments, when introducing the transition metal ions in step (c), introducing the metal chelators to trap the excess transition metal ions in step (d).

In some embodiments, when introducing the transition metal ions in step (c), one or two of the interchain disulfide bonds is (are) reduced.

As used herein, the term “bear”, “bears” or “bearing” refers to have or having.

In some embodiments of the present application, at first, TCEP reduces one of the interchain disulfide bond within the antibody selectively with the transition metal ions, optionally, the second reductant reduces one, two or three of the remaining three interchain disulfide bonds. The antibody with thiol group site-specific modifications, such as the ADC with D1 or the ADC with D2, could be prepared by the method including the step (a) and (b). The antibody with thiol group site-specific modifications, such as the ADC with D1+D6, the ADC with D1+D3, the ADC with D2+D6, the ADC with D2+D3, the ADC with D0+D6, the ADC with D0+D3, the ADC with D2+D2, the ADC with D2+D4, the ADC with D1+D4 or the ADC with D1+D2 could be prepared by the method including the step (a), (b), (c) and (d).

In some embodiments of the present application, the salt refers to acid addition salts or base addition salts.

In some embodiments of the present application, acid addition salts can be formed with inorganic acids and organic acids. The inorganic acids from which salts can be derived include, for example, hydrochloric acid, hydrobromic acid, sulfuric acid, phosphoric acid, and the like. The organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

In some embodiments of the present application, base addition salts can be formed with inorganic bases and organic bases. The inorganic bases from which salts can be derived include groups 1 to 2 of the periodic table. In certain embodiments, the salts are derived from lithium, sodium, potassium, calcium, magnesium and the like. The organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In some embodiments of the present application, the molar ratio of TCEP and said transition metal ions is 1:1 to 1:180, 1:1 to 1:150, 1:1 to 1:130, 1:1 to 1:100, 1:1 to 1:80 or 1:1 to 1:70. In some embodiments of the present application, the molar ratio of TCEP and said transition metal ions is 1:2 to 1:70. In some embodiments of the present application, the molar ratio of TCEP and said transition metal ions is 1:2 to 1:66. In some embodiments of the present application, the molar ratio of TCEP and the transition metal ions is 1:1 to 1:60. In some embodiments of the present application, the molar ratio of TCEP and said transition metal ions is 1:2 to 1:60. In some embodiments of the present application, the molar ratio of TCEP and said transition metal ions is 1:2 to 1:50. In some embodiments of the present application, the molar ratio of TCEP and said transition metal ions is 1:2 to 1:40. In some embodiments of the present application, the molar ratio of TCEP and said transition metal ions is 1:2 to 1:30. In some embodiments of the present application, the molar ratio of TCEP and said transition metal ions is 1:2 to 1:20. In some embodiments of the present application, the molar ratio of TCEP and said transition metal ions is 1:2 to 1:16. In some embodiments of the present application, the molar ratio of TCEP and said transition metal ions is 1:6 to 1:16. In some embodiments of the present application, the molar ratio of TCEP and said transition metal ions is 1:2 to 1:12.

In some embodiments of the present application, the molar ratio of TCEP and said transition metal ions is 1:0.5, 1:0.8, 1:1, 1:2, 1:4, 1:6, 1:8, 1:10, 1:12, 1:14, 1:16, 1:20, 1:25, 1:30, 1:35, 1:40, 1:45, 1:50, 1:55, 1:60, 1:65, 1:70, 1:75, 1:80, 1:85, 1:90, 1:95, 1:100, 1:110, 1:115, 1:120, 1:125, 1:130, 1:140, 1:150, 1:160, 1:170, 1:180, 1:190 or 1:200.

In step (a), TCEP and the transition metal ions together generate selectivity in one of four interchain disulfide bonds reduction. The molar ratio of TCEP and the transition metal ions is very important to selectively reduce one interchain disulfide bond.

In some embodiments of the present application, the molar ratio of the antibody and TCEP is 1:1 to 1:3. In some embodiments of the present application, the molar ratio of the antibody and TCEP is 1:1 to 1:2.5.

In some embodiments of the present application, the molar ratio of the antibody and TCEP is 1:1 to 1:2. In some embodiments of the present application, the molar ratio of the antibody and TCEP is 1:1 to 0.6:1. In some embodiments of the present application, the molar ratio of the antibody and TCEP is 1:1 to 1:1.5. In some embodiments of the present application, the molar ratio of the antibody and TCEP is 1:1 to 1:1.2.

In some embodiments of the present application, the molar ratio of the antibody and TCEP is 1:1. In some embodiments of the present application, the molar ratio of the antibody and TCEP is 1:2, 1:2.1, 1:2.2, 1:2.3, 1:2.4, 1:2.5, 1:2.6, 1:2.7, 1:2.8 or 1:2.9. In some embodiments of the present application, the molar ratio of the antibody and TCEP is 1:1.5. In some embodiments of the present application, the molar ratio of the antibody and TCEP is 1:1.8 or 1:1.9. In some embodiments of the present application, the molar radio of the antibody and TCEP is 0.6:1.

In some embodiments of the present application, there is no specific limitation to the concentration of TCEP in step (a), as long as scaling up or down the concentration of the transition metal ions and the antibody in equal proportions. In some embodiments of the present applications, the concentration of TCEP is 0.01 mM to 0.2 mM. In some embodiments of the present applications, the concentration of TCEP is 0.02 mM to 0.15 mM. In some embodiments of the present applications, the concentration of TCEP is 0.05 mM to 0.1 mM. In some embodiments of the present applications, the concentration of TCEP is 0.01 mM, 0.02 mM, 0.03 mM, 0.04 mM, 0.05 mM, 0.06 mM, 0.07 mM, 0.08 mM, 0.09 mM, 0.10 mM, 0.11 mM, 0.12 mM, 0.13 mM, 0.14 mM, 0.15 mM, 0.16 mM, 0.17 mM, 0.18 mM, 0.19 mM or 0.20 mM.

In some embodiments of the present application, there is no specific limitation to the concentration of the transition metal ions in step (a), as long as scaling up or down the concentration of TCEP and the antibody in equal proportions.

In some embodiments of the present application, there is no specific limitation to the concentration of the antibody in step (a), as long as scaling up or down the concentration of TCEP and the transition metal ions in equal proportions.

In some embodiments of the present application, the first buffer system and the second buffer system are independently selected from a group consisting of HEPES buffer, Histidine buffer, PBS, MES buffer, BES buffer, MOPS buffer, Bis-Tris buffer, Acetate buffer, DIPSO buffer, MOPSO buffer, TES buffer, ACES buffer, TAPSO buffer, PIPES buffer, BTP buffer, HEPPSO buffer, POPSO buffer EPPS buffer or Tris buffer.

In some embodiments of the present application, the first buffer system and the second buffer system are independently selected from a group consisting of Bis-Tris buffer, MOPS buffer, MES buffer, HEPES buffer, PIPES buffer, DIPSO buffer, MOPSO buffer, TES buffer, BES buffer and ACES buffer.

In some embodiments of the present application, the first buffer system and the second buffer system are independently selected from a group consisting of Bis-Tris buffer, MOPS buffer, MES buffer, HEPES buffer, PIPES buffer, DIPSO buffer, MOPSO buffer, TES buffer and BES buffer.

In some embodiments of the present application, the first buffer system and the second buffer system are independently selected from a group consisting of Bis-Tris buffer, MOPS buffer, HEPES buffe, BES buffer, PIPES buffer and MES buffer.

In some embodiments of the present application, the pH value of the first buffer system and the second buffer system is 5.5 to 8.

In some embodiments of the present application, the pH value of the first buffer system and the second buffer system is 5.8 to 7.4, preferably, the pH value of the first buffer system and the second buffer system is 6.7 to 7.4.

In some embodiments of the present application, the pH value of the first buffer system and the second buffer system is 6.0 to 7.4. In some embodiments of the present application, the pH value of the first buffer system and the second buffer system is 6.4 to 7.4. In some embodiments of the present application, the pH value of the first buffer system and the second buffer system is 6.7 to 7.4. In some embodiments of the present application, the pH value of the first buffer system and the second buffer system is independently 5.5, 5.8, 6.0, 6.4, 6.7, 7.0, 7.4 or 8.

In some embodiments of the present application, the first buffer system and the second buffer system are MOPS buffer and the pH value of MOPS buffer is 6.7 to 7.4. In some embodiments of the present application, the first buffer system and the second buffer system are MOPS buffer and the pH value of MOPS buffer is 6.7, 7.0 or 7.4.

In some embodiments of the present application, the first buffer system and the second buffer system are MES buffer and the pH value of MES buffer is 5.8 to 6.7. In some embodiments of the present application, the first buffer system and the second buffer system are MES buffer, and the pH value of MES buffer is 5.8, 6.0, 6.6 or 6.7.

In some embodiments of the present application, the first buffer system and the second buffer system are HEPES buffer and the pH value of HEPES buffer is 6.7. In some embodiments of the present application, the first buffer system and the second buffer system are BES buffer and the pH value of BES buffer is 6.7. In some embodiments of the present application, the first buffer system and the second buffer system are Bis-Tris buffer and the pH value of Bis-Tris buffer is 6.7. In some embodiments of the present application, the first buffer system and the second buffer system are PIPES buffer and the pH value of PIPES buffer is 6.7. In some embodiments of the present application, the first buffer system and the second buffer system are DIPSO buffer and the pH value of DIPSO buffer is 7.4. In some embodiments of the present application, the first buffer system and the second buffer system are MOPSO buffer and the pH value of MOPSO buffer is 7.4. In some embodiments of the present application, the first buffer system and the second buffer system are TES buffer and the pH value of TES buffer is 7.4. In some embodiments of the present application, the first buffer system and the second buffer system are ACES buffer and the pH value of ACES buffer is 7.4.

In some embodiments of the present application, the transition metal ions are selected from a group consisting of Zn²⁺, Cd²⁺, Ni²⁺, Hg²⁺, Mn²⁺, Co²⁺ and the combination thereof.

In some embodiments of the present application, the transition metal ions are selected from a group consisting of Zn²⁺, Cd²⁺, Hg²⁺, Ni²⁺, Co²⁺ and the combination thereof.

In some embodiments of the present application, the transition metal ion is Zn²⁺.

In some embodiments of the present application, there is no specific limitation to the salts of the transition metal ions, as long as the transition metal ions are soluble in the reaction solution so that free transition metal ions can be released in the reaction solution. In some embodiments of the present application, the salts of the transition metal ions are chloride, nitrate, sulfate, acetate, iodide, bromine, formate or tetrafluorborate.

In some embodiments of the present application, the salts of Zn²⁺ are ZnCl₂, Zn(NO₃)₂, ZnSO₄, Zn(CH₃COO)₂, ZnI₂, ZnBr₂, Zinc formate, or zinc tetrafluoroborate. In some embodiments of the present application, the salts of Zn²⁺ are ZnCl₂.

Those skilled in the art should understand that the incubation temperature and incubation time in step (a) depend on specific antibodies to be conjugated. In some embodiments of the present application, the incubation temperature is 0° C. to 37° C., 0° C. to 25° C. or 0° C. to 15° C. in step (a), the incubation time is 0.5 h to 24 h in step (a).

In some embodiments of the present application, the incubation temperature is 0° C. to 25° C. in step (a), the incubation time is 0.5 h to 8 h in step (a).

In some embodiments of the present application, the incubation temperature is 0° C. to 15° C., 0° C. to 10° C., 0° C. to 8° C. or 0° C. to 6° C. in step (a);

In some embodiments of the present application, the incubation time is 0.5 h to 24 h, 0.5 h to 20 h, 0.5 h to 16 h, 0.5 h to 12 h, 0.5 h to 8 h or 0.5 h to 6 h in step (a).

In some embodiments of the present application, the incubation temperature is 0° C. to 15° C. in step (a), the incubation time is 0.5 h to 6 h in step (a). In some embodiments of the present application, the incubation temperature is 4° C. to 24° C. in step (a), the incubation time is 2 h to 16 h in step (a). In some embodiments of the present application, the incubation temperature is 0° C. to 10° C. in step (a), the incubation time is 2 h to 5 h in step (a). In some embodiments of the present application, the incubation temperature is 4° C. in step (a), the incubation time is 4 h in step (a).

In some embodiments of the present application, the molar ratio of the antibody and TCEP is 1:2 to 1:3, and the incubation time is 1 h to 5 h in step (a).

In some embodiments of the present application, the molar ratio of the antibody and TCEP is 1:3, and the incubation time is 1 h to 5 h in step (a).

In some embodiments of the present application, the molar ratio of the antibody and TCEP is 1:3, 1:2.9, 1:2.8, 1:2.7, 1:2.6, 1:2.5, 1:2.4, 1:2.3, 1:2.2, 1:2.1, 1:2, and the incubation time is 1 h, 1.5 h, 2 h, 2.5 h, 3 h, 3.5 h, 4 h or 5 h in step (a).

In some embodiments of the present application, in step (c), there is no specific limitation to the second reductant, as long as the second reductant could reduce the interchain disulfide bonds within the antibody. In some embodiments of the present application, the second reductant is TCEP, Tris(3-hydroxypropyl)phosphine (THPP), or Dithiothreitol (DTT). In some embodiments, the second reductant is TCEP.

In some embodiments of the present application, in step (c), without the transition metal ions, the molar ratio of the second reductant and the antibody is 3:1 to 20:1, 4:1 to 10:1, 5:1 to 9:1, 6:1 to 9:1, 6:1 to 8:1. In some embodiments, the molar ratio of the second reductant and the antibody is 3:1, 4:1, 5:1, 6:1, 7:1, 8:1, 9:1, 10:1, 12:1, 14:1, 16:1, 18:1 or 20:3.

In some embodiments of the present application, the incubation temperature of the second reductant is 0° C. to 37° C., or 5° C. to 30° C. in step (c). In some embodiments of the present application, the incubation temperature of the second reductant is 10° C. to 30° C., 15° C. to 30° C., 20° C. to 30° C., or 25° C. to 30° C. in step (c). In some embodiments of the present application, the incubation temperature of the second reductant is 25° C. in step (c).

In some embodiments of the present application, the incubation time of the second reductant is 0.5 h to 24 h, or 5 h to 20 h in step (c). In some embodiments of the present application, the incubation time of the second reductant is 6 h to 18 h, 8 h to 18 h, 8 h to 15 h, or 8 h to 12 h in step (c). In some embodiments of the present application, the incubation time of the second reductant is 8 h or 12 h in step (c).

In some embodiments, in step (c), introducing the transition metal ions, two of the interchain disulfide bonds are selectively reduced. In some embodiments, in step (c), the molar ratio of the second reductant and the transition metal ions is 1:0.05 to 1:40, and/or the molar ratio of antibody and the second reductant is 1:2.5 to 1:20, and/or the incubation time is 1 h to 24 h. In some embodiments, in step (c), the molar ratio of the second reductant and the transition metal ions is 1:0.05, 1:0.08, 1:0.1, 1:0.2, 1:0.3, 1:0.4, 1:0.5, 1:0.6, 1:0.7, 1:0.8, 1:0.9, 1:1, 1:2, 1:4, 1:6, 1:8, 1:10, 1:12, 1:14, 1:16, 1:18 or 1:20. In some embodiments, in step (c), the molar ratio of the antibody and the second reductant is 1:2.5, 1:3, 1:5, 1:7, 1:9, 1:11, 1:13, 1:15, 1:17, 1:19 or 1:20. In some embodiments, in step (c), the incubation time is 1 h, 2 h, 4 h, 6 h, 8 h, 10 h, 12 h, 14 h, 16 h, 18 h, 20 h, 22 or 24 h. In some embodiments, in step (c), the molar ratio of the second reductant and the transition metal ions is 1:0.05 to 1:40, and/or the molar ratio of the antibody and the second reductant is 1:3 to 1:15, and the incubation time is 2 h to 12 h. In some embodiments, in step (c), the molar ratio of the second reductant and the transition metal ions is 1:0.05 to 1:40, and/or the molar ratio of the antibody and the second reductant and is 1:2.5 to 1:15, and the incubation time is 12 to 24 h.

In some embodiments, introducing the transition metal ions, one of the interchain disulfide bonds are selectively reduced. In some embodiments, in step (c), the molar ratio of the second reductant and the transition metal ions is 1:0.5 to 1:100, and/or the molar ratio of the antibody and the second reductant is 1:0.8 to 1:2.5, and/or the incubation time is 0.5 h to 24 h. In some embodiments, in step (c), the molar ratio of the second reductant and the transition metal ions is 1:0.5, 1:1, 1:4, 1:8, 1:12, 1:24, 1:30, 1:40, 1:50, 1:50, 1:70, 1:80, 1:90, 1:100. In some embodiments, in step (c), the molar ratio of the antibody and the second reductant is 1:0.8, 1:1, 1:1.2, 1:1.4, 1:1.6, 1:1.8, 1:2, 1:2.2, 1:2.4, or 1:2.5. In some embodiments, in step (c), the incubation time is 0.5 h, 1 h, 2 h, 4 h, 6 h, 8 h, 10 h, 12 h, 14 h, 16 h, 18 h, 20 h, 22 or 24 h. In some embodiments, in step (c), the molar ratio of the second reductant and the transition metal ions is 1:0.5 to 1:100, and/or the molar ratio of the antibody and the second reductant is 1:0.8 to 1:2, and the incubation time is 0.5 h to 24 h. In some embodiments, in step (c), the molar ratio of the second reductant and the transition metal ions is 1:0.5 to 1:100, and/or the molar ratio of the antibody and the second reductant is 1:2 to 1:2.5, and the incubation time is 1 h to 9 h.

In some embodiments of the present application, in step (b) and in step (d), the reaction temperature with the reduced thiol groups is 4° C. to 37° C., 20° C. to 30° C. or 20° C. to 25° C. In some embodiments of the present application, in step (b) and in step (d), the reaction temperature with the reduced thiol groups is 24° C.

In some embodiments of the present application, in step (b) and in step (d), the reaction time with the reduced thiol groups is 0.5 h to 6 h, 0.5 h to 5 h, 0.5 h to 4 h, 0.5 h to 2 h or 0.5 h to 1. In some embodiments of the present application, in step (b) and in step (d), the reaction time with the reduced thiol groups is 0.5 h, 1 h, 2 h or 3 h.

In some embodiments of the present application, the reactive temperature and time with the reduced thiol groups in step (b) and step (d) are independent.

In some embodiments of the present application, in step (b) and in step (d), the reaction temperature with the reactive groups is 10° C. to 37° C., 20° C. to 30° C., 10° C. to 30° C., 15° C. to 30° C. or 25° C. to 30° C. In some embodiments, in step (b) and in step (d), the reaction temperature with the reactive groups is 20° C., 22° C., 24° C., 25° C., 27° C. or 29° C.

In some embodiments of the present application, in step (b) and in step (d), the reaction time with the reactive groups is 2 h to 12 h, 2 h to 10 h, 4 h to 10 h, 6 h to 10 h, or 8 h to 10 h. In some embodiments of the present application, in step (b) and (d), the reaction time with the reactive groups is 8 h.

In some embodiments of the present application, the reactive temperature and time with the reactive groups in step (b) and step (d) are independent.

In some embodiments of the present application, the metal chelators can trap excessive said transition metal ions in step (b). In some embodiments of the present application, there is no specific limitation to the metal chelators, as long as the metal chelators can trap the excessive transition metal ions and do not affect the reduction of the disulfide bonds within the antibody. In some embodiments of the present application, the metal chelators are selected from a group consisting of ethylene diamine tetraacetic acid (EDTA), nitrilotriacetic acid (NTA), diethylenetriaminepentaacetic acid (DTPA), citric Acid (CA), tartaric acid (TA), gluconic acid (GA) or N-(2-hydroxyethyl)ethylenediamine-N,N′,N′-triacetic acid (HEDTA).

In some embodiments of the present application, the metal chelators are selected from a group consisting of EDTA, NTA or DTPA. In some embodiments of the present application, the metal chelators are EDTA.

In some embodiments, the molar ratio of the metal chelators and the antibody in step (b) is 1:1 to 100:1, 10:1 to 100:1, 20:1 to 100:1, 20:1 to 80:1, 20:1 to 70:1, 30:1 to 60:1, 40:1 to 50:1, 35:1 to 60:1, 40:1 to 55:1.

In some embodiments, the molar ratio of the metal chelators and the antibody in step (d) is 1:1 to 100:1, 1:1 to 60:1, 1:1 to 50:1, 1:1 to 20:1, 1:1 to 10:1, 1:1 to 8:1, 1:1 to 6:1, 1:1 to 5:1, 2:1 to 8:1, 2:1 to 6:1.

In some embodiments of the present application, the excess amount of metal chelators and a complex of the metal chelators and the transition metal ions are filtered out in dialysis, ultrafiltration or gel filtration.

In some embodiments of the present application, in step (b), according to the amount of the antibody, the modification reagent 1 is excess.

In some embodiments of the present application, in step (b), the molar ratio of the first thiobridge reagent and the antibody is 5:1 to 1:1, 2:1 to 1:1, 1.5:1 to 1:1, 1.2:1 to 1:1 or 1.1:1 to 1:1. In some embodiments of the present application, in step (b), the molar ratio of the first thiobridge reagent and the antibody is 1.05:1.

In some embodiments of the present application, in step (b), when the first linker-payload reacts with the reduced thiol groups, the molar ratio of the first linker-payload and the antibody is 2:1 to 10:1, 3:1 to 10:1, 4:1 to 9:1 or 5:1 to 7:1. In some embodiments, in step (b), when the first linker-payload reacts with the reduced thiol groups, the molar ratio of the first linker-payload and the antibody is 5:1.

In some embodiments of the present application, in step (b), when the first linker-payload reacts with the reactive groups in the first thiobridge reagent, the molar ratio of the first linker-payload and the antibody is 5:1 to 1:1, 4:1 to 1:1.1, 3:1 to 1:1 or 2:1 to 1:1. In some embodiments, when the first linker-payload reacts with the reactive groups in the first thiobridge reagent, in the step (b), the molar ratio of the first linker-payload and the antibody is 5:3.

In some embodiments of the present application, in step (d), according to the amount of the antibody, the modification reagent 2 is excess.

In some embodiments of the present application, in step (d), the molar ratio of the second thiobridge reagent and the antibody is 5:1 to 1:1, 5:1 to 3:1, 4:1 to 3:1, 4:1 to 3.2:1 or 4:1 to 3.5:1.

In some embodiments of the present application, in step (d), when the second linker-payload reacts with the reduced thiol groups, the molar ratio of the second linker-payload and the antibody is 20:1 to 2:1, 20:1 to 6:1, 18:1 to 8:1, 16:1 to 8:1, 14:1 to 8:1, 12:1 to 10:1. In some embodiments of the present application, in step (d), when the second linker-payload reacts with the reduced thiol groups, the molar ratio of the second linker-payload and the antibody is 35:3.

In some embodiments of the present application, in step (d), when the second linker-payload reacts with the reactive groups in the second thiobridge reagent, the molar ratio of the second linker-payload and the antibody is 10:1 to 1:1, 10:1 to 2:1, 10:1 to 3:1, 9:1 to 3:1, 8:1 to 3:1, 7:1 to 3:1, 6:1 to 3:1, 5:1 to 3:1 or 4:1 to 3:1.

In some embodiments of the present application, said method further comprises the following steps:
optionally, introducing a compound that contains at least one thiol group to consume excessive said first linker-payload in step (b) and/or said second linker-payload in step (d);
purifying and recovering the resultant antibody with thiol group site-specific modifications in step (b) and/or in step (d).

In some embodiments of the present application, there is no specific limitation to a compound to consume excessive said first linker-payload and/or said second linker-payload, as long as the compound contains at least one thiol group. In some embodiments of the present application, the compound is cysteine.

By purifying and recovering the resultant antibody with thiol group site-specific modifications in step (b) and/or in step (d), the content of the antibody with thiol group site-specific modifications could be higher. In some embodiments of the present application, the resultant antibody with thiol group site-specific modifications is purified by a de-salting column, size exclusion chromatography, ultrafiltration, dialysis and/or the like. In some embodiments of the present application, the resultant antibody with thiol group site-specific modifications is purified by a de-salting column. If needed, further enrichment (e.g., D2) may be applied in some case using hydrophobic interaction chromatography (HIC).

In some embodiments of the present application, there is no specific limitation to the antibody. According to the antigens associated with the disease, those skilled in the art can select suitable antibody useful in the bio-conjugation process of the present application. In some embodiments of the present application, the antibody is a monoclonal antibody, a polyclonal antibody, a mono-specific antibody or a multi-specific antibody.

In some embodiments of the present application, the antibody is a human antibody, a humanized antibody, a chimeric antibody or an antigen-binding moiety thereof.

In some embodiments of the present application, the antibody means an immunoglobulin and is a molecule containing an antigen-binding site immunospecifically binding to an antigen. In some embodiments of the present application, the class of the antibody is IgG, IgE, IgM, IgD, IgA, or IgY. In some embodiments of the present application, the class of the antibody is IgG.

In some embodiments of the present application, the class of the antibody is IgG1, IgG2, IgG3 or IgG4. In some embodiments of the present application, the antibody is. IgG1 or IgG4. In some embodiments of the present application, the antibody is IgG1.

In some embodiments of the present application, the antibody comprises at least one mutation in the Fc region. In some embodiments, the at least one mutation modulates effector function, or attenuates or eliminates Fc-g receptor binding.

In some embodiments of the present application, the one or more mutations are to stabilize the antibody and/or to increase half-life. In some instances, the one or more mutations are to modulate Fc receptor interactions, to reduce or eliminate Fc effector functions such as FcyR, antibody-dependent cell-mediated cytotoxicity (ADCC), or complement-dependent cytotoxicity (CDC). In additional instances, the one or more mutations are to modulate glycosylation.

In some embodiments of the present application, the one or more mutations are located in the Fc region. In some instances, the Fc region comprises a mutation at residue position L234, L235, or a combination thereof. In some instances, the mutations comprise L234 and L235. In some instances, the mutations comprise L234A and L235A. In some cases, the residue positions are in reference to IgG1.

In some embodiments of the present application, the Fc region comprises a mutation at residue position L234, L235, D265, N21, K46, L52, or P53, or a combination thereof. In some instances, the mutations comprise L234 and L235 in combination with a mutation at residue position K46, L52, or P53.

In some embodiments of the present application, the Fc region comprises mutations at L234, L235, and K46. In some cases, the Fc region comprises mutations at L234, L235, and L52. In some cases, the Fc region comprises mutations at L234, L235, and P53. In some cases, the Fc region comprises mutations at D265 and N21. In some cases, the residue position is in reference to IgG1.

In some embodiments of the present application, the Fc region comprises L234A, L235A, D265A, N21G, K46G, L52R, or P53G, or a combination thereof. In some instances, the Fc region comprises L234A and L235A in combination with K46G, L52R, or P53G. In some cases, the Fc region comprises L234A, L235A, and K46G. In some cases, the Fc region comprises L234A, L235A, and L52R. In some cases, the Fc region comprises L234A, L235A, and P53G. In some cases, the Fc region comprises D265A and N21G. In some cases, the residue position is in reference to IgG1.

In some embodiments of the present application, the Fc region comprises a mutation at residue position L233, L234, D264, N20, K45, L51, or P52. In some instances, the Fc region comprises mutations at L233 and L234. In some instances, the Fc region comprises mutations at L233 and L234 in combination with a mutation at residue position K45, L51, or P52. In some cases, the Fc region comprises mutations at L233, L234, and K45. In some cases, the Fc region comprises mutations at L233, L234, and L51. In some cases, the Fc region comprises mutations at L233, L234, and K45. In some cases, the Fc region comprises mutations at L233, L234, and P52. In some instances, the Fc region comprises mutations at D264 and N20. In some cases, equivalent positions to residue L233, L234, D264, N20, K45, L51, or P52 in an IgG1, IgG2, IgG3, or IgG4 framework are contemplated.

In some embodiments of the present application, the Fc region comprises L233A, L234A, D264A, N20G, K45G, L51R, or P52G. In some instances, the Fc region comprises L233A and L234A. In some instances, the Fc region comprises L233A and L234A in combination with K45G, L51R, or P52G. In some cases, the Fc region comprises L233A, L234A, and K45G. In some cases, the Fc region comprises L233A, L234A, and L51R. In some cases, the Fc region comprises L233A, L234A, and K45G. In some cases, the Fc region comprises L233A, L234A, and P52G. In some instances, the Fc region comprises D264A and N20G.

In some embodiments of the present application, the human IgG constant region is modified to alter antibody-dependent cellular cytotoxicity (ADCC) and/or complement-dependent cytotoxicity (CDC), e.g., with an amino acid modification described in Natsume et al., 2008 Cancer Res, 68(10): 3863-72; Idusogie et al., 2001 J Immunol, 166 (4): 2571-5; Moore et al., 2010 mAbs, 2 (2): 181-189; Lazar et al, 2006 PNAS, 103(11): 4005-4010, Shields et al, 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kancko and Niwa, 2011 Biodrugs, 25(1): 1-11.

In some embodiments of the present application, the antibody of IgG1, IgG2, IgG3 or IgG4 is human or humanized antibody. The information of IgG1, IgG2, IgG3 or IgG4 can be obtained on NCBI or UniProt (https://www.uniprot.org/).

In some embodiments of the present application, the antibody is bispecific antibodies. In some embodiments of the present application, the antibody is IgG1 like bispecific antibodies.

In some embodiments of the present application, those skilled in the art can select suitable method to prepare the bispecific antibodies. In some embodiments of the present application, the bispecific antibodies can be obtained by Knobs-in-holes technology (Ridgway J B B, Presta L G, Paul C. ‘Knobs-into-holes’ engineering of antibody CH3 domains for heavy chain heterodimerization[J]. Protein Engineering(7):617(2023 Aug. 11).), format chain exchange (FORCE) technology, a common light chain format technology (De Nardis C, Hendriks L J A, Poirier E, et al. A new approach for generating bispecific antibodies based on a common light chain format and the stable architecture of human immunoglobulin G1[J]. Journal of Biological Chemistry, 2017:jbc. M117.793497), controlled Fab arm exchange technology (Yanakieva De, Pekar L, Evers A, et al. Beyond bispecificity: Controlled Fab arm exchange for the generation of antibodies with multiple specificities[J]. MABS, 2022, 14(1), c2018960), CrossMAb technology (Klein C, Schaefer W, Regula J T. The use of CrossMAb technology for the generation of bi- and multispecific antibodies[J]. MABS, 2016, 8(6), P1010-P1020) or their combination.

As used herein, the term “knobs-into-holes” is used in its broadest sense and encompasses various situations, such as the CH1 domain of one heavy chain with the knob mutations and the CH1 domain of the other heavy chain with the hole mutations, the CH2 domain of one heavy chain with the knob mutations and the CH2 domain of the other heavy chain with the hole mutations, and/or the CH3 domain of one heavy chain with the knob mutations and the CH3 domain of the other heavy chain with the hole mutations. For example, and generally, “knobs-into-holes” may refer to an intra-interface modification between two antibody heavy chains in the CH3 domains: i) in the CH3 domain of one heavy chain (first CH3 domain), an amino acid residue is substituted with another amino acid residue bearing a large side chain, thereby creating a protrusion (“knob”) in the interface in the first CH3 domain; ii) in the CH3 domain of the other heavy chain (second CH3 domain), an amino acid residue is substituted with another amino acid residue bearing a smaller side chain, thereby creating a cavity (“hole”) within the interface in the second CH3 domain, in which a protrusion (“knob”) in the first CH3 domain can be placed.

In some embodiments of the present application, the antibody is selected from any one of cytotoxic antibodies, inhibitors of cell proliferation, regulators of cell activation and interaction, regulators of the human immune system, neutralizations of antigens, antibodies that are immunospecific for viral antigens or antibodies that are immunospecific for microbial antigens.

In some embodiments of the present application, the antibody can be target-specific antibodies, In some embodiments of the present application, without the limitation, the antibody can be anti-HER2 antibody, anti-FAP antibody, anti-OX-40 antibody, anti-41BB antibody, anti-Angiopoietin-2 antibody, anti-ant-IL-4Rα antibody, anti-BCMA antibody, anti-Blys antibody, anti-BTNO2 antibody, anti-C5 antibody, anti-CD122 antibody, anti-CD13 antibody, anti-CD133 antibody, anti-CD137 antibody, anti-CD138 antibody, anti-CD16a antibody, anti-CD19 antibody, anti-CD20 antibody, anti-CD22 antibody, anti-CD27 antibody, anti-CD28 antibody, anti-CD3 antibody, anti-CD30 antibody, anti-CD33 antibody, anti-CD38 antibody, anti-CD40 antibody, anti-CD47 antibody, anti-CD-8 antibody, anti-CD79 antibody, anti-CEA antibody, anti-CGPR/CGRPR antibody, anti-CSPGs antibody, anti-CTLA4 antibody, anti-CTLA-4 domains antibody, anti-DLL-4 antibody, anti-EGFR antibody, anti-EpCAM antibody, anti-factor IXa antibody, anti-factor X antibody, anti-GITR antibody, anti-GP130 antibody, anti-Her3 antibody, anti-HSG antibody, anti-ICOS antibody, anti-IGF1 antibody, anti-IGF1/2 antibody, anti-IGF-IR antibody, anti-IGF2 antibody, anti-IGFR antibody, anti-IL-1 antibody, anti-IL-12 antibody, anti-IL-12p40 antibody, anti-IL-13 antibody, anti-IL-17A antibody, anti-IL-1β antibody, anti-IL-23 antibody, anti-IL-5 antibody, anti-IL-6 antibody, anti-IL-6R antibody, anti-Lag-3 antibody, anti-LAG3 antibody, anti-MAG antibody, anti-Met antibody, anti-NgR antibody, anti-NogoA antibody, anti-OMGp antibody, anti-OX40 antibody, anti-PD-1 antibody, anti-PDGFR antibody, anti-PDL-1 antibody, anti-PSMA antibody, anti-RGMA antibody, anti-RGMB antibody, anti-SARS-COV-2 antibody, anti-Te38 antibody, anti-TIM-3 antibody, anti-TNF antibody, anti-TNFα antibody, anti-TROP-2 antibody, anti-TWEAK antibody, anti-VEGF antibody, or anti-VEGFR antibody.

In some embodiments of the present application, the antibody can be Transtuzumab, Sacituzumab, Belantamab, Risankizumab, Eptinezumab, Teprotumumab, Polatuzumab, Tafasitamab, Rovelizumab, Romosozumab, Dostarlimab, Enfortumab or Ublituximab.

In some embodiments of the present application, the antibody can be Transtuzumab, Sacituzumab or Belantamab.

In some embodiments of the present application, the antibody can be obtained commercially or produced by any method known to those skilled in the art.

In some embodiments of the present application, the first thiobridge reagent and the second thiobridge reagent independently contain at least two substituted groups allowing a re-bridging of the thiol groups.

In some embodiments of the present application, without the limitation, the first thiobridge reagent and the second thiobridge reagent are independently selected from the group consisting of

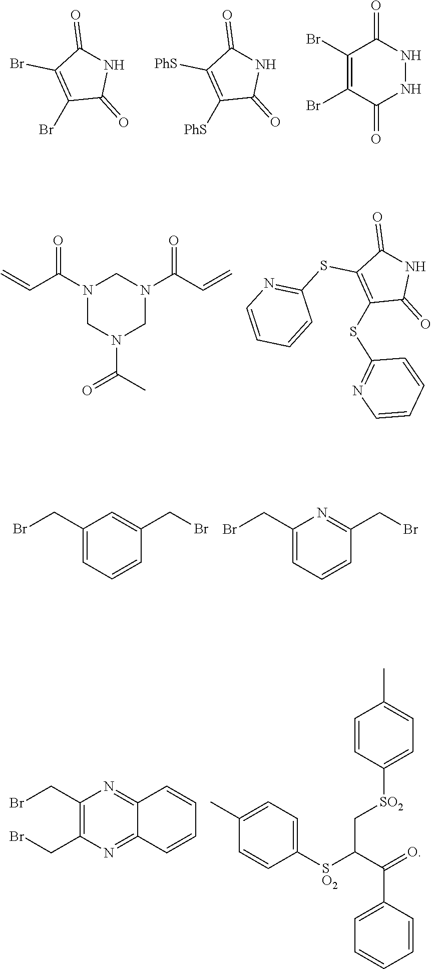

In some embodiments of the present application, the reactive groups independently contain azido and/or dibenzocyclooctyne (DBCO).

In some embodiments of the present application, the thiobridge reagent and the reactive groups are connected by alkyl group or polyethylene glycol (PEG).

In some embodiments of the present application, without the limitation, the first thiobridge reagent bearing reactive groups and the second thiobridge reagent bearing reactive groups are independently selected from the groups consisting of

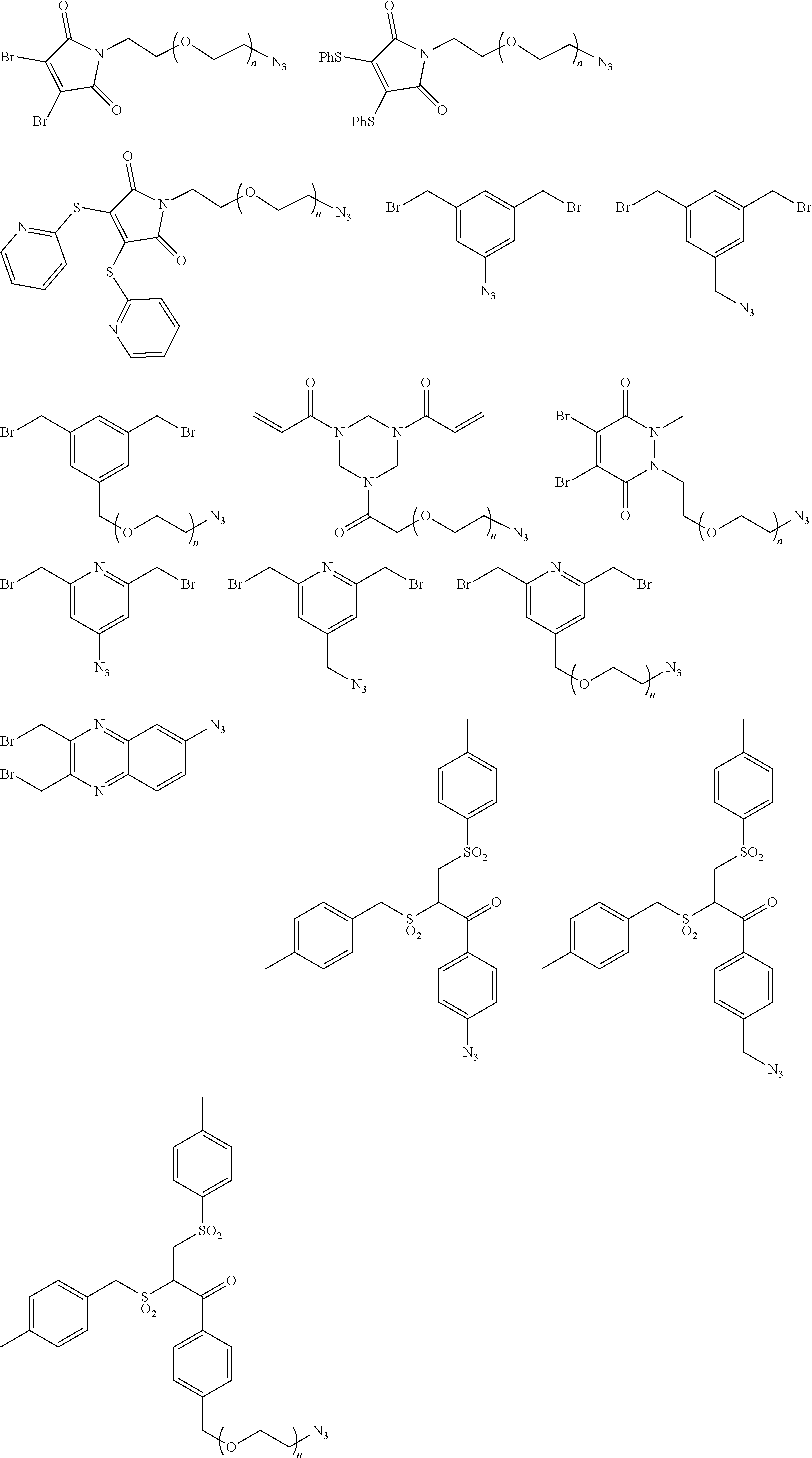

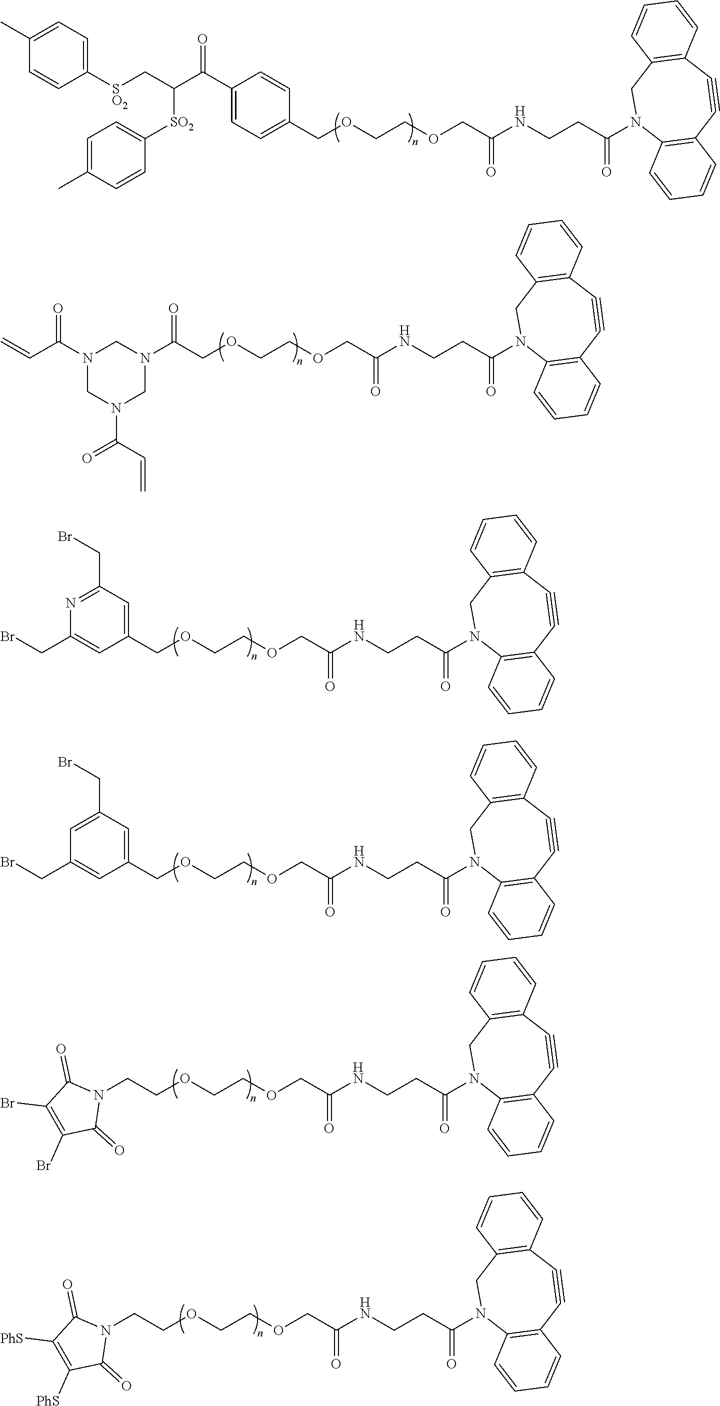

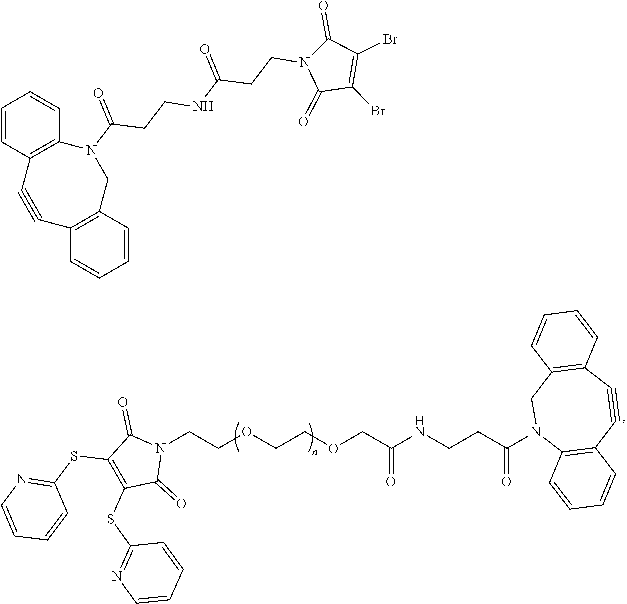

wherein, n is 0-20, 0-18, 0-15, 0-13, 0-10, 0-7, 0-5 or 0-3, optionally, n is 0, 1, 2, 3, 4, 5, 6, 7, 8, 9 or 10.

In some embodiments of the present application, the first thiobridge reagent bearing reactive groups and the second thiobridge reagent bearing reactive groups are dibromomaleimide-PEG4-N3, having the following formula

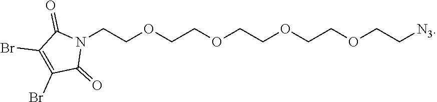 [SMILES: [N-]=[N+]=NCCOCCOCCOCCOCCN1C(=O)C(Br)=C(Br)C1=O]

In some embodiments of the present application, a linker of the first linker-payload and the second linker-payload is selected from any one of which the one terminal can be connected to the reduced thiol group of the antibody or the reactive groups of the thiobridge reagent, and the other terminal can be connected to the payload.

In some embodiments of the present application, when the first linker-payload and/or the second linker-payload react(s) with the reduced thiol groups, the linker of the first linker-payload and the second linker-payload independently includes a cleavable linker or a noncleavable linker. Cleavable linkers can be chemically labile and enzyme-labile linkers. Due to the high plasma stability and good intracellular cleaving selectivity and efficiency, enzyme-labile linkers are broadly selected as cleavable linker candidates in ADCs. In some embodiments, enzyme-labile linkers comprise the structure: -maleimidocaproyl- (-MC-), -maleimidocaproyl-peptide moiety- (-MC-peptide moiety-), -p-aminobenzyl alcohol- (-PAB-), or -peptide moiety-. In some embodiments of the present application, the peptide moiety is dipeptides, tripeptides, tetrapeptides or pentapeptides.

In some embodiments of the present application, without the limitation, the dipeptides can be valine-alanine (VA), valine-citrulline (VC), alanine-asparagine (AD), alanine-phenylalanine (AF), phenylalanine-lysine (FK), alanine-lysine (AK), alanine-valine (AV), valine-lysine (VK), lysine-lysine (KK), phenylalanine-citrulline (FC), leucine-citrulline (LC), isoleucine-citrulline (IC), tryptophan-citrulline (WC) or phenylalanine-alanine (FA).

In some embodiments of the application, without the limitation, the tripeptides can be alanine-alanine-asparagine (AAD), glycine-valine-citrulline (GVC), glycine-glycine-glycine (GGG), phenylalanine-phenylalanine-lysine (FFK), glutamic acid-valine-citrulline (EVC), or glycine-phenylalanine-lysine (GFK).

In some embodiments of the application, without the limitation, the tetrapeptides can be glycine-glycine-phenylalanine-glycine (GGFG).

In some embodiments of the present application, without the limitation, when the first linker-payload and/or the second linker-payload react(s) with the reduced thiol groups, the linker of the first linker-payload and the second linker-payload can be MC-VA-PAB, MC-VC-PAB, MC-AD-PAB, MC-AF-PAB, MC-FK-PAB, MC-AK-PAB, MC-AV-PAB, MC-VK-PAB, MC-KK-PAB, MC-FC-PAB, MC-LC-PAB, MC-IC-PAB, MC-WC-PAB or MC-FA-PAB independently. In some embodiments of the present application, without the limitation, when the first linker-payload and/or the second linker-payload react(s) with the reduced thiol groups, the linker of the first linker-payload and the second linker-payload can be MC-AAD-PAB, MC-GVC-PAB, MC-GGG-PAB, MC-FFK-PAB, MC-EVC-PAB, or MC-GFK-PAB independently.

In some embodiments of the present application, when the first linker-payload and/or the second linker-payload react(s) with the reactive groups in the thiobridge reagent, the linker of the first linker-payload and/or the second linker-payload further include(s) azido and/or dibenzocyclooctyne (DBCO)). In some embodiments of the present application, when the linker of the first linker-payload and/or the second linker-payload contains azido, the reactive groups of the thiobridge group contain DBCO. In some embodiments, when the linker of the first linker-payload and/or the second linker-payload contains DBCO, the reactive groups of the thiobridge group contain azido.

In some embodiments of the present application, when the first linker-payload and/or the second linker-payload react(s) with the reactive groups in the thiobridge reagent, the linker of the first linker-payload and the second linker-payload is independently selected from any one of the groups consisting of

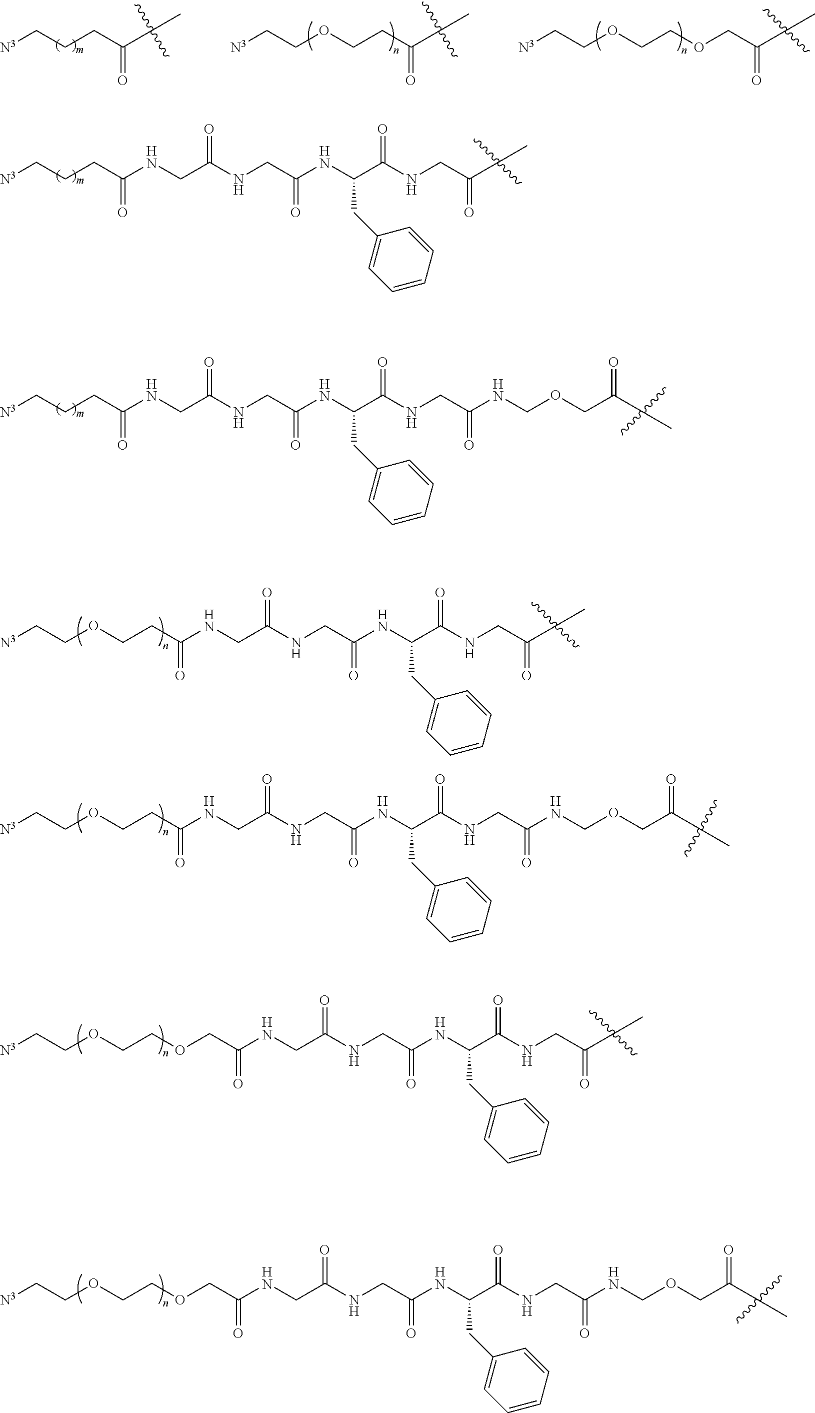

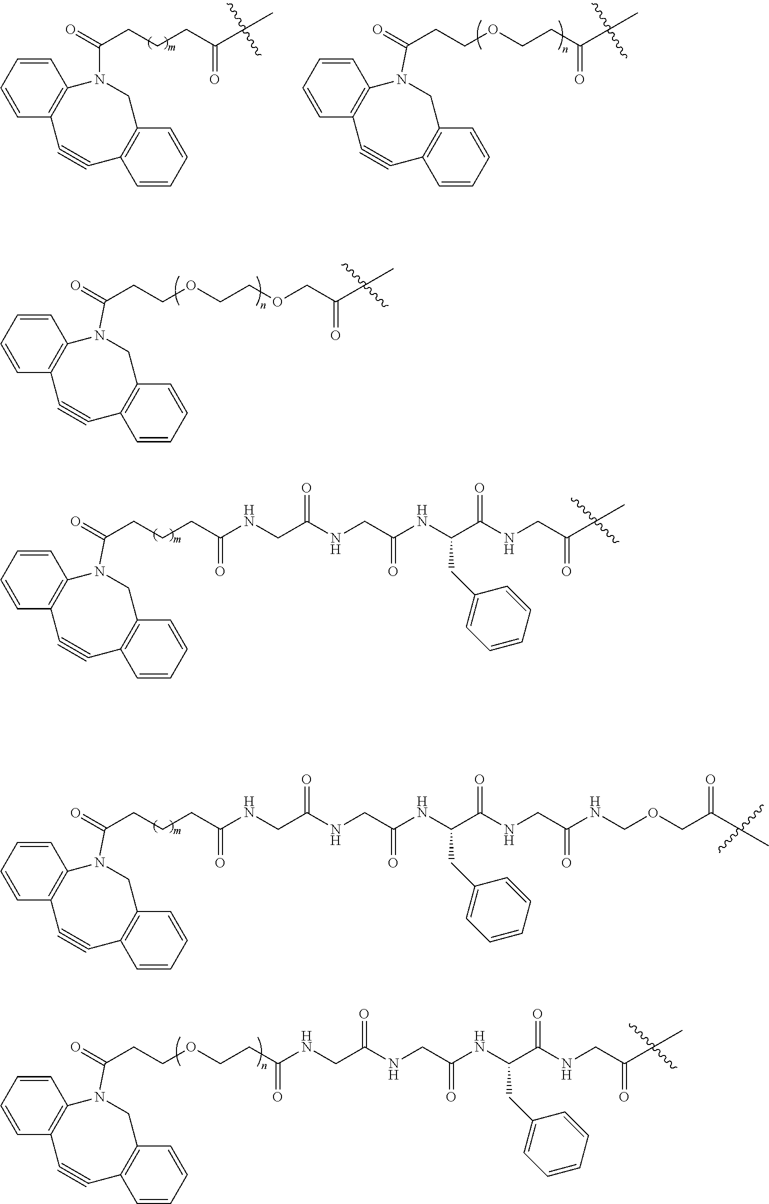

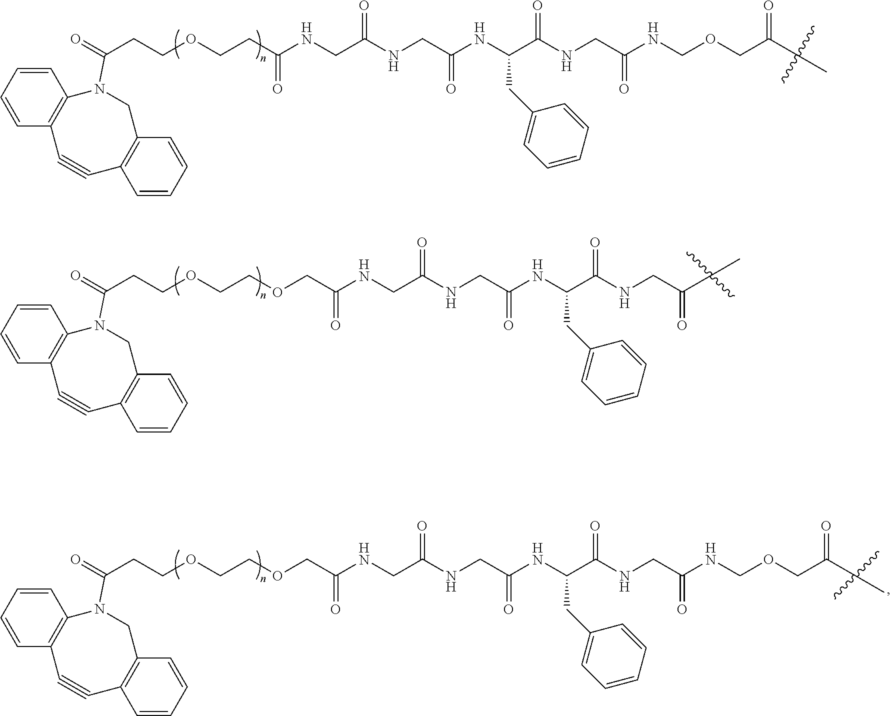

wherein, n is 0-20, 0-18, 0-15, 0-13, 0-10, 0-7, 0-5 or 0-3, m is 0-20, 0-18, 0-15, 0-13, 0-10, 0-7, 0-5 or 0-3, optionally, n is 0, 1, 2, 3, 4, 5, 6, 7, 8, 9 or 10, m is 0, 1, 2, 3, 4, 5, 6, 7, 8, 9 or 10.

As used herein, the term “end capping reagent” refers to a compound which does not bear a drug and contains at least one substituted group which can covalently couple to an antibody.

In some embodiments, the end capping reagent is the cleavable linker or the noncleavable linker. In some embodiments, the end capping reagent is (2-Aminoethyl) maleimide.

In some embodiments of the present application, there is no specific limitation to the payload, as long as the payload contains at least one substituted group allowing a connection from the payload to the linker.

In some embodiments of the present application, the payload is a cytotoxic drug, a fluorescent dye, a cytokine, a nucleic acid, a radionuclide, a kinase inhibitor or derivatives thereof. In some embodiments of the present application, the payload includes but not limited to topoisomerases inhibitor and tubulin inhibitors. In some embodiments of the present application, without the limitation, the payload can be anti-cancer agent, antiviral agent or antimicrobial agent.

In some embodiments of the present application, the cancer is carcinoma, lymphoma, blastema, sarcoma, and leukemia or lymphoid malignancies. More particular examples of the cancer include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

In some embodiments of the present application, without the limitation, the payload can be monomethyl auristatin E (MMAE), monomethyl auristatin D (MMAD), monomethyl auristatin EF (MMAF), calicheamicins (CLM), mertansine (DM1), maytansinoids, duocarmycins, anthracyclines, pyrrolobenzodiazepine dimers, amatoxin, quinolinealkaloid, DXd, doxorubicin hydrochloride, methotrexate, erlotinib, bortezomib, fulvestrant, sunitib imatinib mesylate, letrozole, finasunate, platins such as oxaliplatin, carboplatin, and cisplatin, finasunate, fluorouracil, rapamycin, leucovorin, lapatinib, lonafamib, sorafenib, gefitinib, camptothecin, topotecan, bryostatin, adozelesin, anthracycline, carzelesin, bizelesin, dolastatin, auristatins, duocarmycin, eleutherobin, taxols such as paclitaxel or docetaxel, cyclophosphamide, doxorubicin, vincristine, prednisone or prednisolone, other alkylating agents such as mechlorethamine, chlorambucil, and ifosfamide, antimetabolites such as azathioprine or mercaptopurine, vincristine, vinblastine, vinorelbine, vindesine, etoposide, teniposide, etoposide phosphate, epipodophyllotoxins, actinomycin, daunorubicin, valrubicin, idarubicin, edrecolomab, epirubicin bleomycin, plicamycin or mitomycin.

In some embodiments of the present application, the payload is deruxtecan (DXd), cyanine 3 (Cy3), MMAE, MMAD or MMAF. In some embodiments of the present application, the payload is MMAE, DXd or Cy3.

The linker-payload is a chemical moiety, which is synthesized by connecting the linker to the payload. Depending on the desired payload and selected linker, those skilled in the art can select suitable method for coupling them together. For example, some conventional coupling methods, such as amine coupling methods, may be used to form the desired linker-payload which still contains substituted groups for conjugating to the antibodies through covalent linkage. A drug-maleimide complex (i.e., maleimide linking drug) is taken as an example of the linker-payload in the present disclosure. Most common group capable of bonding to thiol group in ADC preparation is maleimide. Additionally, organic chloride, bromides, iodides also are frequently used.

In some embodiments of the present application, when the first linker-payload and/or the second linker-payload react(s) with the reduced thiol groups, the first linker-payload and/or the second linker-payload are independently MC-VC-PAB-MMAE, MC-VC-PAB-MMAD or MC-VC-PAB-MMAF.

In some embodiments of the present application, the first thiobridge reagent bearing the first linker-payload and the second thiobridge reagent bearing the second linker-payload independently have the following formula:

$$Q - S - T ,$$

wherein, Q is selected from the groups consisting of

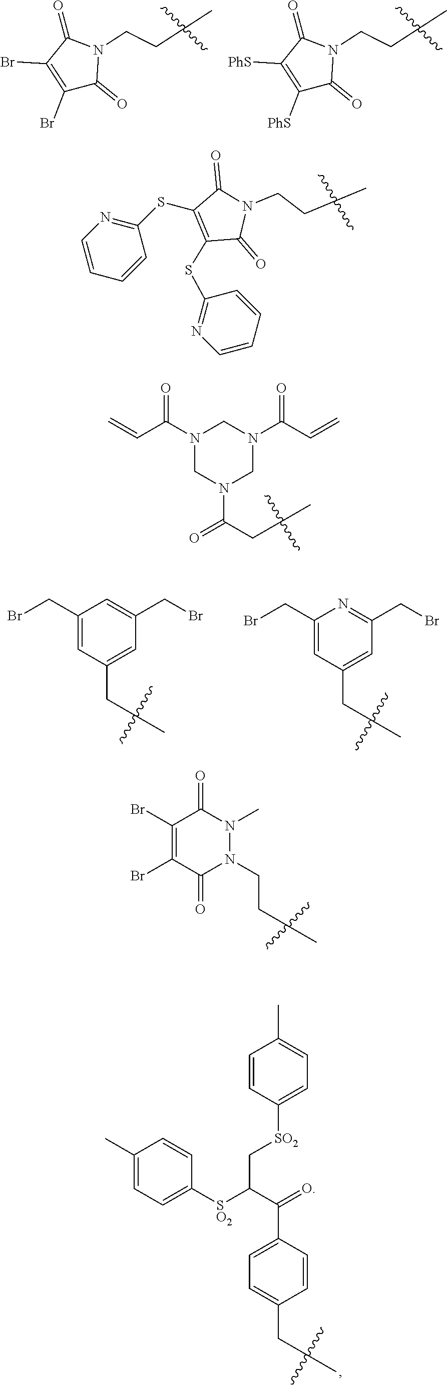

S is selected from a cleavable linker or a non-cleavable linker, without the limitation. S is selected from the groups consisting of

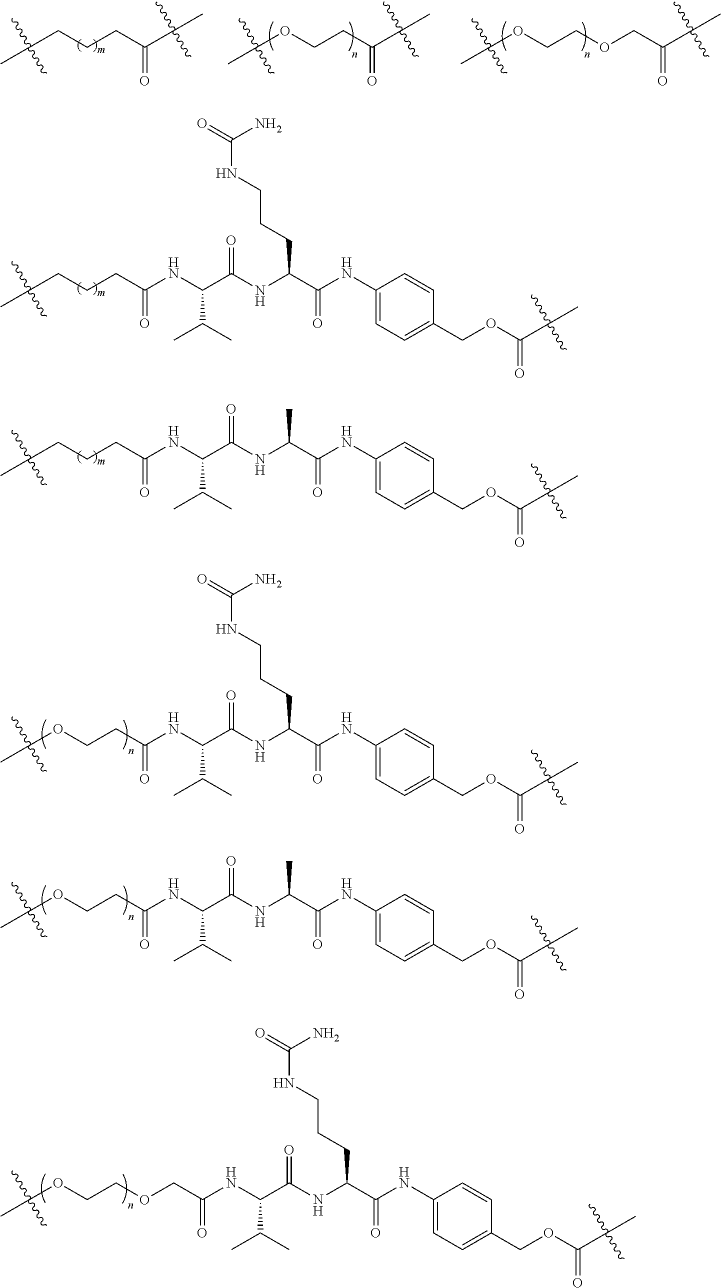

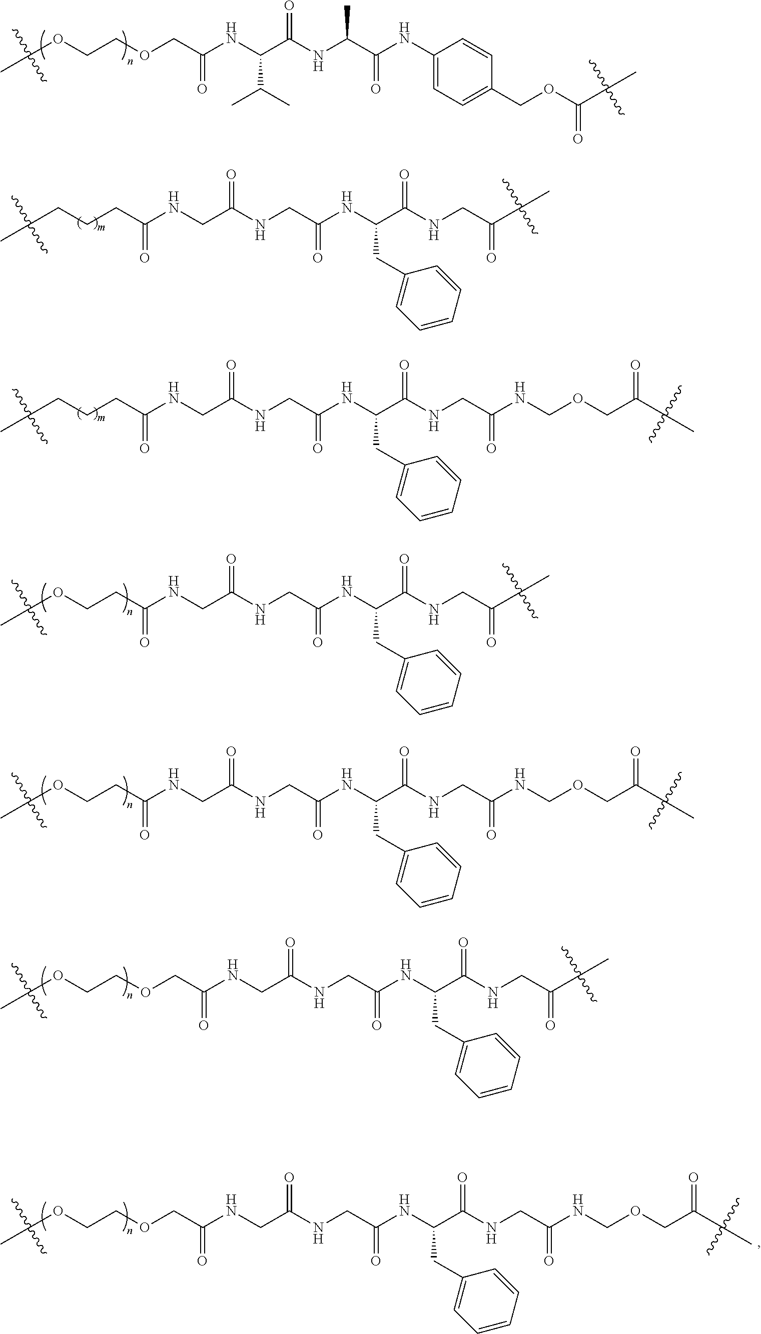

Wherein, n is 0-20, m is 0-20, optionally, n is 0, 1, 2, 3, 4, 5, 6, 7, 8, 9 or 10, m is 0, 1, 2, 3, 4, 5, 6, 7, 8, 9 or 10,
T is payload.

In some embodiments of the present application, without the limitation, the first thiobridge reagent bearing the first linker-payload and the second thiobridge reagent bearing the second linker-payload are independently selected from the group consisting of

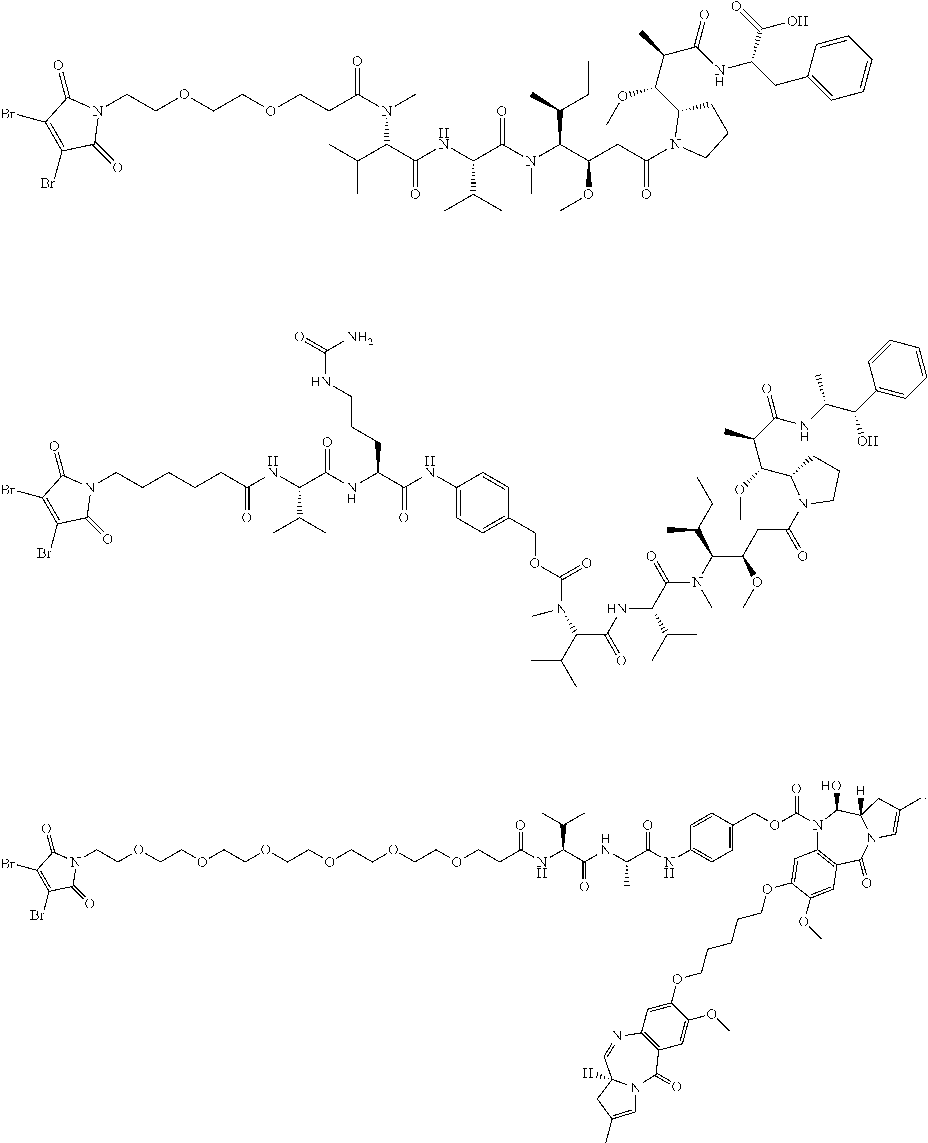

In some embodiments of the present application, the payload of the first thiobridge reagent bearing the first linker-payload and that of the second thiobridge reagent bearing the second linker-payload are different or same. In some embodiments, the linker of the first thiobridge reagent bearing the first linker-payload and that of the second thiobridge reagent bearing the second linker-payload could be different or same. In some embodiments, the thiobridge reagent of the first thiobridge reagent bearing the first linker-payload and that of the second thiobridge reagent bearing the second linker-payload could be different or same.

In some embodiments of the present application, said method of preparing the ADC with D2 comprises the following steps:
(a1) incubating TCEP or the salt thereof and the antibody in the presence of an effective amount of the transition metal ions in the first buffer system to selectively reduce one of four interchain disulfide bonds within the antibody, the molar ratio of TCEP and said transition metal ions is 1:1 to 1:70, optionally, the molar ratio of TCEP and said transition metal ions is 1:2 to 1:16, more optionally, the molar ratio of TCEP and said transition metal ions is 1:12;
(b1) introducing an excess amount of the metal chelators and an excess amount of the first linker-payload to react with the reduced thiol groups resulted from step (a1).

In some embodiments of the present application, the homogeneity of the ADC with D2 is up to 55%, 60%, 65%, 69%, 70%, 72%, 74%, 76%, 78%, 80% or 82%, even to 83.

In some embodiments of the present application, said method of preparing the ADC with D2+D6 comprises the following steps:
(c2) incubating the reaction product from step (b1) and the second reductant in the second buffer system to reduce the interchain disulfide bonds within the reaction product from step (b1);
(d2) introducing the incubation product from step (c2) and an excess amount of the second linker-payload to react with the reduced thiol groups resulted from step (c2).

In some embodiments of the present application, the homogeneity of the ADC with D2+D6 is up to 65%, 70%, 72%, even to 75%.

In some embodiments of the present application, the method of preparing the ADC with D2+D3 comprises the following steps:
(d3) introducing the incubation product from step (c2) and an excess amount of the second thiobridge reagent bearing the second linker-payload to react with the reduced thiol groups resulted from the (c2).

In some embodiments of the present application, the method of preparing the ADC with D2+D3 comprises the following the steps:
(d3′) introducing the incubation product from step (c2) and an excess amount of the second thiobridge reagent bearing reactive groups to re-bridge the reduced thiol groups resulted from step (c2), then, incubating an excess amount of the second linker-payload in the second buffer system to react with the reactive groups of the thiobridge group.

In some embodiments of the present application, said method of preparing the ADC with D1 comprises the following steps:
(b4) introducing an excess amount of the metal chelators and an excess amount of the first thiobridge reagent bearing the first linker-payload to react with the reduced thiol groups resulted from step (a1).

In some embodiments of the present application, the method of preparing the ADC with D1 comprises the following the steps:
(b4) introducing an excess amount of the metal chelators and the first thiobridge reagent bearing reactive groups to re-bridge the reduced thiol groups resulted from step (al), then, incubating an excess amount of the first linker-payload in the first buffer system to react with the reactive groups of the thiobridge group.

In some embodiments of the present application, the homogeneity of the ADC with D1 is up to 65%, 70%, 75%, even to 77% or 80%.

In some embodiments of the present application, said method of preparing the ADC with D1+D6 comprises the following steps:
(c5) incubating the reaction product from step (b4) or step (b4) and the second reductant in the second buffer system to reduce the interchain disulfide bonds within the reaction product from step (b4) or (b4);
(d5) introducing the incubation product from step (c5) and an excess amount of the second linker-payload to react with the reduced thiol groups resulted from step (c5).

In some embodiments of the present application, the method of preparing the ADC with D1+D3 comprises the following steps:
(d6) introducing the incubation product from step (c5) and an excess amount of the second thiobridge reagent bearing the second linker-payload to react with the reduced thiol groups resulted from the (c5).

In some embodiments of the present application, the method of preparing the ADC with D1+D3 comprises the following the steps:
(d6′) introducing the incubation product from step (c5) and an excess amount of the second thiobridge reagent bearing reactive groups to re-bridge the reduced thiol groups resulted from step (c5), then, incubating an excess amount of the second linker-payload in the second buffer system to react with the reactive groups of the thiobridge group.

In some embodiments of the present application, the method of preparing the ADC with D0+D6 comprises the following steps:
(b7) introducing an excess amount of the metal chelators and an excess amount of the first thiobridge reagent to react with the reduced thiol groups resulted from step (a1).
(c7) incubating the reaction product from step (b7) and the second reductant in the second buffer system to reduce the interchain disulfide bonds within the reaction product from step (b7);
(d7) introducing the incubation product from step (c7) and an excess amount of the second linker-payload to react with the reduced thiol groups resulted from step (c7).

In some embodiments of the present application, the homogeneity of the ADC with D0+D6 is up to 65%, 70%, 73%, even to 75%.

In some embodiments of the present application, the method of preparing the ADC with D0+D3 comprises the following steps:
(d8) introducing the incubation product from step (c7) and an excess amount of the second thiobridge reagent bearing the second linker-payload to react with the reduced thiol groups resulted from the (c7).

In some embodiments of the present application, the method of preparing the ADC with D0+D3 comprises the following the steps:
(d8′) introducing the incubation product from step (c7) and an excess amount of the second thiobridge reagent bearing reactive groups to re-bridge the reduced thiol groups resulted from step (c7), then, incubating an excess amount of the second linker-payload in the second buffer system to react with the reactive groups of the thiobridge group.

In some embodiments, the antibody with thiol group site-specific modification (ADC with D2+D2, ADC with D2+D4) is prepared by the method with step (a), (b), (c) and (d), wherein the modification reagent 1 and the modification reagent 2 are the linker-payloads. Meanwhile, the transition metal ions are introduced in step (c).

In some embodiments, the antibody with site-specific modification (ADC with D1+D2, D1+D4) is prepared by the method with step (a), (b), (c) and (d), wherein the modification reagent 1 is the first thiobridge reagent bearing the first linker-payload, or the modification reagent 1 is the first thiobridge reagent bearing reactive groups which reacts with the first linker-payload, and the modification reagent 2 is the second linker-payload.

Various analytical methods can be used to determine the yields and isomeric mixtures of the ADC. In some embodiments of the present application, the analytical method is HIC-HPLC. HIC-HPLC is able to separate the ADC which antibodies loaded with various numbers of drugs. The drug loading level can be determined based on the ratio of absorbances, e.g., at 250 nm and 280 nm. For example, if a drug can absorb at 250 nm while the antibody absorbs at 280 nm. The 250/280 ratio therefore increases with drug loading.

As compared with ADCs generated by conventional conjugation processes, using the bio-conjugation process described herein, the ADCs of the present application have improved homogeneity without need of protein engineering, without need of ligases, and has simple manipulation and reduced cost.

In some embodiments of the present application, the method of preparing ADC with D2 comprises e following steps:
(1) Incubating TCEP (0.02 mM) and Transtuzumab (0.012 mM) in the presence of ZnCl₂ (0.24 M) in MES buffer (pH6.7, 20 mM). The incubation temperature is 4° C. and the incubation time is 4 h;
(2) EDTA was added to trap Zn²⁺;
(3) Introducing MC-VC-PAB-MMAE (0.06 mM) to react with reduced thiol groups resulted from step (1), the reaction temperature is 24° C. and the reaction time is 0.5 h;
(4) Introducing cysteine (0.08 mM) to consume excessive MC-VC-PAB-MMAE;
(5) The resulted ADC was subjected to purification using a de-salting column.
   An Antibody with Thiol Group Site-Specific Modifications

On the second aspect, the present application provides an antibody with thiol group site-specific modifications prepared by the method of the present application.

In some embodiments of the present application, the antibody with thiol group site-specific modifications is conjugated with the modification reagent 1 and/or the modification reagent 2.

In some embodiments of the present application, the antibody with thiol group site-specific modifications is conjugated with the modification reagent 1, forming the ADC with D2 or the ADC with D1. In some embodiments of the present application, the antibody with thiol group site-specific modifications is conjugated with the modification reagent 1 and the modification reagent 2, forming the ADC with D2+D6, the ADC with D2+D3, the ADC with D1+D6, the ADC with D1+D3, the ADC with D0+D6, the ADC with D0+D3, the ADC with D2+D2, the ADC with D2+D4, the ADC with D1+D2 or the ADC with D1+D4.

In some embodiments of the present application, the ADC with D2 is Transtuzumab-[MC-VC-PAB-MMAE]₂, Sacituzumab-[MC-VC-PAB-MMAE]₂ and/or Belantamab-[MC-VC-PAB-MMAE]₂.

In some embodiments of the present application, the ADC with D1 is Trastuzumab-[Maleimide-PEG4-N3-DBCO-Cy3]₁, the ADC with D0+D6 is Trastuzumab-[Maleimide]₁[MC-VC-PAB-MMAF]₆ and the ADC with D2+D6 is Trastuzumab-[MC-VC-PAB-MMAE]₂[MC-GGFG-DXd]₆.

### Pharmaceutical Composition

On the third aspect, the present application provides a pharmaceutical composition comprising the antibody with thiol group site-specific modifications according to the present application and one or more of pharmaceutically acceptable carrier.

In some embodiments of the present application, there is no specific limited to the pharmaceutically acceptable carrier. The choice of the pharmaceutically acceptable carrier to a large extent depends on the factors such as the particular mode of administration, the effect of the carrier on solubility and the stability, and the nature of the dosage form. In some embodiments of the present, the pharmaceutically acceptable carrier includes any and all solvent, coating, isotonic agent, absorption delaying agent, wetting agent, viscosifier, pH regulator, stabilizer, surfactant, antioxidants, diluents, binders, disintegrants, buffers, preservatives, lubricants, flavorings, thickeners, coloring agents, emulsifiers and the like that are physiologically compatible.

In some embodiments of the present application, the solvent is water, dextrose, glycerol, ethanol and the like.

In some embodiments of the present application, the isotonic agent is sugars, polyalcohol (e.g., mannitol, sorbitol), sodium chloride and the like.

In some embodiments of the present application, the viscosifier is sodium hyaluronate, kambo, sodium carboxymethyl cellulose, methyl cellulose, polyethylene glycol, polyvinyl alcohol, povidone and the like.

In some embodiments of the present application, the pH regulator is sodium dihydrogen phosphate, disodium hydrogen phosphate, potassium dihydrogen phosphate, potassium dihydrogen phosphate, boric acid, acetic acid, sodium acetate, citric acid, sodium citrate, tartaric acid, sodium tartrate, sodium carbonate, potassium carbonate, sodium bicarbonate, potassium bicarbonate, sodium hydroxide, potassium hydroxide, hydrochloric acid, phosphoric acid and the like.

In some embodiments of the present application, the stabilizer is disodium edilate, calcium disodium edilate, dipotassium edilate, diamine edilate, a-lipoic acid, ethylene glycol dimethacrylate, sodium oleate, anhydrous sodium sulfite, sodium ascorbate, desferric amine, malate, citric acid, succinate, sodium, calcium and magnesium salts of malate, citric acid, succinate and the like.

In some embodiments of the present application, the surfactant is poloxam, sodium dodecyl sulfate, Tween-20, Tween-40, Tween-60, Tween-65, Tween-80, Tween-85, lecithin, polyoxyethylene castor oil, polyoxyethylene hydrogenated castor oil, polyvinylpyrrolidone, polyethylene glycols, polyethylene glycol 15-hydroxystearate and the like.

In some embodiments of the present application, the antioxidant is methionine, ascorbic acid, EDTA, sodium thiosulfate, platinum, catalase, citric acid, cysteine, thioglycerol, thioglycolic acid, thiosorbitol, butylated hydroxyanisole, butylated hydroxytoluene, and/or propyl gallate and the like. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine.

In some embodiments of the present application, the pharmaceutical compositions provided herein may be formulated in any manner known in the art. the pharmaceutical composition can be formulated for particular routes of administration such as oral administration, parenteral administration and rectal administration. Topical administration may also pertain to inhalation or intranasal application. the pharmaceutical compositions of the present invention can be made up in a solid form (including, without limitation, capsules, tablets, pills, granules, powders or suppositories), or in a liquid form (including, without limitation, solutions, suspensions or emulsions). In some embodiments of the present application, the pharmaceutical compositions can be a liquid solution, suspension, or emulsion. In some embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In some embodiments of the present application, the pharmaceutical composition is suitable for parenteral administration. The parenteral administration of the pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. In some embodiments of the present application, the pharmaceutical composition is administration of the pharmaceutical composition by injection of the pharmaceutical composition, by application of the pharmaceutical composition through a surgical incision, by application of the pharmaceutical composition through a tissue-penetrating non-surgical wound and the like. In some embodiments of the application, the parenteral administration is subcutaneous, intraperitoneal, intermuscular, intrasternal, intravenous, intraarterial, intrathecal, intraventricular, intracranial, intrasynovial injection, infusions or kidney dialytic infusion techniques.

In some embodiments of the present application, the pharmaceutical composition is combined with other therapeutic agents. There is no specific limitation to the other therapeutic agents, as long as the other therapeutic agents can reduce the side effects of the pharmaceutical composition or increase the efficacy of the pharmaceutical composition. The other therapeutic agents are anti-cancer agents, anti-autoimmune disease agent, anti-emetics, anti-allergic and the like.

In some embodiments of the present application, the anti-cancer agents can include, but not limited to, erlotinib, bortezomib, fulvestrant, sunitib imatinib, mesylate, letrozole, finasunate, platins such as oxaliplatin, carboplatin, and cisplatin, finasunate, fluorouracil, rapamycin, leucovorin, lapatinib, lonafamib, sorafenib, gefitinib, camptothecin, topotecan, bryostatin, adozelesin, anthracycline, carzelesin, bizelesin, dolastatin, auristatins, duocarmycin, eleutherobin, taxols such as paclitaxel or docetaxel, cyclophosphamide, doxorubicin, vincristine, prednisone or prednisolone, other alkylating agents such as mechlorethamine, chlorambucil, and ifosfamide, antimetabolites such as azathioprine or mercaptopurine, other microtubule inhibitors (vinca alkaloids like vincristine, vinblastine, vinorelbine and vindesine, as well as taxanes), podophyllotoxins (etoposide, teniposide, etoposide phosphate, and cpipodophyllotoxins), topoisomerase inhibitors, other cytotoxins such as actinomycin, daunorubicin, valrubicin, idarubicin, epirubicin, bleomycin, plicamycin, mitomycin and the like.

In some embodiments of the present application, the anti-autoimmune disease agent can include, but not limited to, ibuprofen, loxoprofen, naproxen, diclofenac, indomethacin, meloxicam, lornoxicam, nabumetone, celecoxib, paracetamol, glucocorticoids, azathioprine, cyclophosphamide and the like.

In some embodiments of the present application, patients may experience nausea during and after administration of the ADCs of the present application. Therefore, anti-emetics may be administered in preventing nausea (upper stomach) and vomiting. The anti-emetics can include, but not limited to, aprepitant, ondansetron, granisetron HCl, lorazepam, dexamethasone, prochlorperazine, casopitant and the like.

In some embodiments of the present application, patients may experience allergic reactions during and after administration of the ADCs of the present application. Therefore, anti-allergic agents may be administered to minimize the risk of an allergic reaction. The anti-allergic agents include dexamethasone, beclomethasone, hydrocortisone, prednisolone, prednisone, methylprednisolone, hydroxyzine, cyproheptadine, bronchodilators, terbutaline and the like.

### Use of TCEP

On the fourth aspect, the present application provides use of TCEP or a salt thereof in the preparation of the antibody with thiol group site-specific modifications according to the present application.

In some embodiments of the present application, TCEP and the transition metal ions are used together.

In the present application, in some embodiments, TCEP and the transition metal ions together selectively reduce one of four inter-chain disulfide bonds of the antibody. And thus, the modification reagent 1 is attached to the antibody to form the ADC with D1, the ADC with D2 or the ADC with D0 with high homogeneity, optionally, the second reductant is introduced to reduce the other interchain disulfide bonds within the antibody and the modification reagent 2 is introduced to modify the remaining reduced thiol groups to form the ADC with D1+D6, the ADC with D1+D3, the ADC with D2+D6, the ADC with D2+D3, the ADC with D0+D6 or the ADC with D0+D6 with high homogeneity.

Use of the Antibody with Thiol Group Site-Specific Modifications

On the fifth aspect, the present application provides use of the antibody with thiol group site-specific modifications according to the present application in the manufacture of a therapeutic agent for diagnosing, preventing or treating a disease.

In some embodiments of the present application, the disease may be cancer, autoimmune disease and the like. In some embodiments of the present application, the disease is cancer.

In some embodiments of the present application, the cancer can include, but not limited to, carcinoma, lymphoma, blastema, sarcoma, and leukemia or lymphoid malignancies. More particular examples of the cancer include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

On the sixth aspect, the present application provides a method of preventing or treating a disease in a subject in need thereof, comprising administrating to the subject a therapeutically effective amount of the antibody with thiol group site-specific modifications according to the present application.

In some embodiments of the present application, the subject in need can be human.

In some embodiments of the present application, the therapeutically effective amount will vary with the type and severity of the condition to be alleviated. It is to be further understood that for any particular subject, specific dosage regimens should be adjusted over time according to the individual need and the professional judgment of the person administering or supervising the administration of the compositions. In some embodiments of the present application, the therapeutically effective amount is based on a variety of factors, such as the type of disease, the age, weight, sex, medical condition of the patient, the severity, of the condition, the route of administration, and the particular antibody employed. In some embodiments of the present application, the therapeutically effective amount can vary widely, but can be determined routinely using standard methods. In some embodiments of the present application, the therapeutically effective amount can be adjusted based on the pharmacokinetic or pharmacodynamic parameters, which may include clinical effects such as toxic effects and/or laboratory values.

It will be readily apparent to those skilled in the art that other suitable modifications and adaptations of the present application described herein are obvious and may be made using suitable equivalents without departing from the scope of the disclosure or the embodiments disclosed herein. Having now described the disclosure in detail, the same will be more clearly understood by reference to the following examples, which are included for purposes of illustration only and are not intended to be limiting. Further, unless specifically described otherwise, the reagent and the solvent described in the description can be easily obtained from a commercial supplier.

### EXAMPLES

### Example 1: Preparation of Transtuzumab-[MC-VC-PAB-MMAE]₂ Conjugate by Using the Process of the Present Application (the ADC with D2)

(1) ZnCl₂ (0.24 mM) and TCEP (commercially available from Bidepharm, 0.02 mM) were added to a solution of Transtuzumab (commercial available from Roche, 0.012 mM, in MES buffer, commercially available from Macklin, pH6.7, 20 mM) and the reaction mixture was allowed to stay at 4° C. for 4 h;
(2) EDTA (commercially available from Aladdin, 0.6 mM) was added to trap Zn²⁺;
(3) MC-VC-PAB-MMAE (0.06 mM, commercially available from Levena biopharma) in DMA (commercially available from Aladdin) was introduced and the reaction was continued at 24° C. for 30 min;
(4) cysteine (commercially available from Aladdin, 0.08 mM) was added to deplete excessive MC-VC-PAB-MMAE;
(5) The reaction mixture was subjected to purification using a de-salting column (Thermo, type: 40K, 0.5 mL, REF: 87766, Lot SJ251704).

### Example 2: Preparation of Sacituzumab-[MC-VC-PAB-MMAE]₂ Conjugate by Using the Process of the Present Application (the ADC with D2)

The method of example 2 is the same as example 1, and the difference is that trastuzumab of example 1 is replaced by Sacituzumab (commercially available from MedChemExpress) of example 2.

### Example 3: Preparation of Belantamab-[MC-VC-PAB-MMAE]₂ Conjugate by Using the Process of the Present Application (the ADC with D2)

The method of example 3 is the same as example 1, and the difference is that trastuzumab of example 1 is replaced by Belantamab (commercially available from MedChemExpress) of example.

### Examples 4-16 and Comparative Example 4: Preparation of Transtuzumab-[MC-VC-PAB-MMAE]₂ Conjugate (the ADC with D2) (the Molar Ratio of TCEP and Zn²⁺ is Different)

The method of examples 4-16 and comparative example 4 is similar to example 1, and the difference is that the concentration of ZnCl₂ in step (1).

TABLE 1
the concentration of Zn²⁺ of
examples 4-12 and comparative example 4

| | | |
|---|---|---|
| | | the molar ratio of |
| No. | the concentration of Zn²⁺ | TCEP and Zn²⁺ |

| | | | |
|---|---|---|---|
| E4 | 0.008 | mM | 1:0.4 |
| E5 | 0.02 | mM | 1:1 |
| E6 | 0.04 | mM | 1:2 |
| E7 | 0.08 | mM | 1:4 |
| E8 | 0.12 | mM | 1:6 |
| E9 | 0.16 | mM | 1:8 |
| E10 | 0.20 | mM | 1:10 |
| E11 | 0.24 | mM | 1:12 |
| E12 | 0.28 | mM | 1:14 |
| E13 | 0.32 | mM | 1:16 |
| E14 | 0.6 | mM | 1:30 |
| E15 | 1.4 | mM | 1:70 |
| E16 | 2.5 | mM | 1:125 |
| C4 | 5 | mM | 1:250 |

“E” was short for Example. “C” was short for comparative example.

### Example 17-20: Preparation of Trastuzumab-[MC-VC-PAB-MMAE]₂ Conjugate with Different Molar Ratio of the Antibody and the TCEP

The preparation of Trastuzumab-[MC-VC-PAB-MMAE]₂ conjugate is similar to the example 1, but it adjusts the dosage of the antibody in step (1) or the incubation time in step (1). The dosage of antibody and the molar ratio of the antibody and the TCEP are as follows:

| | The antibody | The antibody/TCEP | Intubation time |
|---|---|---|---|
| No. | (mM) | (Molar Ratio) | in step (1) (h) |
| E17 | 0.017 | 1:1.2 | 4 |
| E18 | 0.01 | 1:2.0 | 1 |
| E19 | 0.008 | 1:2.5 | 1 |
| E20 | 0.0067 | 1:3.0 | 1 |

### Examples 21-34: Preparation of Transtuzumab-[MC-VC-PAB-MMAE]₂ Conjugate by Using the Different Buffer System

The method of examples 21-34 is similar to example 1, and the difference is that the MES buffer of example 1 is replaced by different buffer (The buffers of examples 21-34 are commercially available from Macklin) of examples 21-34.

TABLE 2
the buffer system and the pH value of
the buffer system of examples 21-34

| | | The pH value of |
|---|---|---|
| No. | The buffer system | the buffer system |
| Example 21 | MOPS buffer | 6.7 |
| Example 22 | MOPS buffer | 7.0 |
| Example 23 | MOPS buffer | 7.4 |
| Example 24 | Bis-Tris buffer | 6.7 |
| Example 25 | PIPES buffer | 6.7 |
| Example 26 | BES buffer | 6.7 |
| Example 27 | MES buffer | 6.7 |
| Example 28 | HEPES buffer | 6.7 |
| Example 29 | DIPSO buffer | 7.4 |
| Example 30 | MOPSO buffer | 7.4 |
| Example 31 | TES buffer | 7.4 |
| Example 32 | ACES buffer | 7.4 |
| Example 33 | MES buffer | 5.8 |
| Example 34 | MES buffer | 6.4 |

### Examples 35-44: Preparation of Trastuzumab-[MC-VC-PAB-MMAE]₂ Conjugate (the Reduction Time or Temperature in Step (1) is Different)

The method of examples 35-44 is similar to example 1, and the difference is the reduction time or temperature in step (1) which is shown follows. Meanwhile, the conjugation time in step (3) is 1 h in examples 38-44.

| | The reduction temperature | The reduction time |
|---|---|---|
| No. | in step (1) (° C.) | in step (1) (h) |
| E35 | 15 | 4 |
| E36 | 24 | 4 |
| E37 | 37 | 4 |
| E38 | 4 | 1 |
| E39 | 4 | 2 |
| E40 | 4 | 3 |
| E41 | 4 | 4 |
| E42 | 4 | 5 |
| E43 | 4 | 6 |
| E44 | 4 | 8 |

### Example 45: Preparation of Trastuzumab-[Maleimide-PEG4-N3-DBCO-Cy3]₁ (the ADC with D1)

### 1. Synthesis of Dibromomaleimide-PEG4-N3

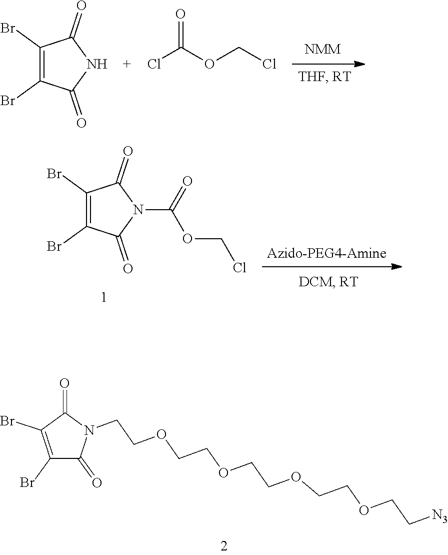 [SMILES: O=C1NC(=O)C(Br)=C1Br ; O=C(Cl)OCCl ; O=C(OCCl)N1C(=O)C(Br)=C(Br)C1=O ; [N-]=[N+]=NCCOCCOCCOCCOCCN1C(=O)C(Br)=C(Br)C1=O]

To a solution of 3,4-dibromomaleimide (127 mg, 0.5 mmol, 1 eq) and N-methylmorpholine (0.22 mL, 2 mmol, 4 eq) in THF (3.5 mL), chloromethyl chloroformate (0.18 mL, 2 mmol, 4 eq) was added and the mixture was stirred for 20 min at room temperature. Then DCM (10 mL) was added, the organic phase was washed with H₂O, dried over MgSO₄ and the solvent removed in vacuo to yield the title product 1 (139 mg, 0.4 mmol, 80%).

A solution of Azido-PEG4-Amine (105 mg, 0.4 mmol, 1 eq, Xi'an Confluore Biological Technology Co., Ltd) in dichloromethane (2 mL) was added to a stirred solution of product 1 (139 mg, 0.4 mmol, 1 eq) in dichloromethane (2 mL).

After 30 minutes, dichloromethane (6 mL) was added and the solution washed with a 0.68 M acetate buffer pH 5 (10 mL), water (1 mL), and dried with MgSO₄. Concentration in vacuo followed by purification by column chromatography (100% EtOAc as the mobile phase) yielded dibromomaleimide-PEG4-N3 (the title product 2) as a pale yellow oil (150 mg, 0.3 mmol, 75%).

### 2. Preparation of Trastuzumab-[Maleimide-PEG4-N3-DBCO-Cy3]₁

(1) incubating TCEP (0.02 mM) and trastuzumab (0.012 mM) in the presence of an effective amount of ZnCl₂ (0.24 mM) in MES (20 mM, pH6.7), The incubation temperature is 4° C. and the incubation time is 4 h;
(2) introducing EDTA (0.6 mM) and dibromomaleimide-PEG4-N3 (0.013 mM) to react with reduced thiol groups resulted from step (1), the reaction temperature is 24° C. and the reaction time is 3 h, then recovering the product using a desalting column to afford Trastuzumab-[Maleimide-PEG4-N3]₁;
(3) incubating Trastuzumab-[Maleimide-PEG4-N3]₁ and DBCO-Cy3 (0.02 mM) in MES (20 mM, pH6.7), the reaction temperature is 25° C. and the reaction time is 8 h, then recovering Trastuzumab-[Maleimide-PEG4-N3-DBCO-Cy3]₁ using a desalting column.

### Example 46: Preparation of Trastuzumab-[Maleimide]₁[MC-VC-PAB-MMAE]₆ (the ADC with D0+D6)

(1) incubating the first reductant TCEP (0.02 mM) and trastuzumab (0.012 mM) in the presence of an effective amount of ZnCl₂ (0.24 mM) in MES (20 mM, pH6.7), The incubation temperature is 4° C. and the incubation time is 4 h;
(2) introducing EDTA (0.6 mM) and the first thiobridge reagent dibromomaleimide (0.013 mM) to react with reduced thiol groups resulted from step (1), the reaction temperature is 24° C. and the reaction time is 3 h, then recovering the product using a desalting column to afford Trastuzumab-[Maleimide]₁;
(3) introducing Trastuzumab-[Maleimide]₁ and the second reductant TCEP (0.08 mM) in MES (20 mM, pH6.7), the reaction temperature is 25° C.′ and the reaction time is 12 h;
(4) introducing the second linker-payload MC-VC-PAB-MMAE (0.14 mM) to solution from step (3), and the reaction mixture was allowed to stay at 24° C. for 1 h, then recovering Trastuzumab-[Maleimide]₁[MC-VC-PAB-MMAE]₆ using a desalting column.

### Example 47: Preparation of Trastuzumab-[MC-VC-PAB-MMAE]₂[MC-GGFG-DXd]₆ (the ADC with D2+D6)

(1) incubating the first reductant TCEP (0.02 mM) and trastuzumab (0.012 mM) in the presence of an effective amount of ZnCl₂ (0.24 mM) in MES (20 mM, pH6.7), The incubation temperature is 4° C. and the incubation time is 4 h;
(2) introducing EDTA (0.6 mM) and an excess amount of the first linker-payload MC-VC-PAB-MMAE (0.06 mM) to react with reduced thiol groups resulted from step (1), the reaction temperature is 24° C. and the reaction time is 1 h, then recovering the product using a desalting column to afford Trastuzumab-[MC-VC-PAB-MMAF]₂;
(3) introducing Trastuzumab-[MC-VC-PAB-MMAF]₂ and the second reductant TCEP (0.08 mM) in MES (20 mM, pH6.7), the reaction temperature is 25° C. and the reaction time is 8 h;
(4) introducing the second linker-payload MC-GGFG-DXd (0.14 mM, commercially available from Levena biopharma) to solution from step (3), and the reaction mixture was allowed to stay at 24° C. for 1 h, then recovering the resultant Trastuzumab-[MC-VC-PAB-MMAE]₂[MC-GGFG-DXd]₆ using a desalting column.

### Comparative Example 1: Preparation of Transtuzumab-[MC-VC-PAB-MMAE]₂ Conjugate without the Transition Metal Ions

(1) TCEP (0.02 mM) was added to a solution of Transtuzumab (0.012 mM, in MES buffer, pH6.7, 20 mM) and the reaction mixture was allowed to stay at 4° C. for 4 h;
(2) MC-VC-PAB-MMAE (0.06 mM) in DMA (commercially available from Aladdin) was introduced and the reaction was continued at 24° C. for 30 min;
(3) cysteine (0.08 mM) was added to deplete excessive MC-VC-PAB-MMAE;
(4) The reaction mixture was subjected to purification using a de-salting column (Thermo, type: 40K, 0.5 mL, REF: 87766, Lot SJ251704).

### Comparative Example 2: Preparation of Sacituzumab-[MC-VC-PAB-MMAE]₂ Conjugate without the Transition Metal Ions

The method of comparative example 2 is the same as comparative example 1, and the difference is that Transtuzumab of comparative example 1 is replaced by Sacituzumab of comparative example 2.

### Comparative Example 3: Preparation of Belantamab-[MC-VC-PAB-MMAE]₂ Conjugate without the Transition Metal Ions

The method of comparative example 3 is the same as comparative example 1, and the difference is that Transtuzumab of comparative example 1 is replaced by Belantamab of comparative example 3.

### Comparative Examples 5-12: Preparation of Transtuzumab-[MC-VC-PAB-MMAE]₂ Conjugate by Using the Different Buffer System

The method of comparative examples 5-12 is similar to example 1, and the difference is that the MES buffer of example 1 is replaced by the different buffer (commercially available from Macklin) system of comparative examples 5-12.

TABLE 3
the buffer system and the pH value of the
buffer system of comparative examples 5-12

| | | The pH value of |
|---|---|---|
| No. | The buffer system | the buffer system |
| Comparative Example 5 | PB buffer | 5.8 |
| Comparative Example 6 | PB buffer | 6.2 |
| Comparative Example 7 | PB buffer | 6.7 |
| Comparative Example 8 | PB buffer | 7.0 |
| Comparative Example 9 | PB buffer | 7.4 |
| Comparative Example 10 | ADA buffer | 6.7 |
| Comparative Example 11 | MOBS buffer | 7.4 |
| Comparative Example 12 | TAPSO buffer | 7.4 |

### The Performance Test

### 1. Homogeneity Assays

The ADCs distribution were analyzed using HIC-HPLC (Agilent1200) with a TSK gel Butyl-NPR column (4.6 mm IDX 3.5 cm) (commercially available from Tosoh Biosciences) at a flow rate of 0.5 mL/min at 30° C. Solvent A was 1.5 M (NH₄)₂SO₄ and 50 mM potassium phosphate pH 7. Solvent B was 75% v/v 50 mM potassium phosphate pH 7 and 25% v/v isopropanol. The washout procedure is as follows:

| Time [min] | Solvent A [%] | Solvent B [%] |
|---|---|---|
| 0.00 | 100.0 | 0.0 |
| 2.00 | 100.0 | 0.0 |
| 15.00 | 0.0 | 100.0 |
| 17.00 | 0.0 | 100.0 |
| 18.00 | 100.0 | 0.0 |
| 20.00 | 100.0 | 0.0 |

### Results and Discussions

TABLE 4
the results of homogeneity assays of examples 1-3 and comparative examples 1-3

| | | | The molar ratio of | | | | | |
|---|---|---|---|---|---|---|---|---|
| No. | Antibody | Zn²⁺ | Zn²⁺ and TCEP | D0(%) | D2(%) | D4(%) | D6(%) | D8(%) |
| Example 1 | transtuzumab | Yes | 12:1 | 5.22 | 80.6 | 14.17 | 0 | 0 |
| Example 2 | sacituzumab | Yes | 12:1 | 7.77 | 76.5 | 15.74 | 0 | 0 |
| Example 3 | belantamab | Yes | 12:1 | 8.14 | 82.17 | 9.69 | 0 | 0 |
| Comparative Example 1 | transtuzumab | No | / | 32.5 | 48.98 | 18.52 | 0 | 0 |
| Comparative Example 2 | sacituzumab | No | / | 32.82 | 49.08 | 18.1 | 0 | 0 |
| Comparative Example 3 | belantamab | No | / | 19.12 | 48.43 | 32.45 | 0 | 0 |

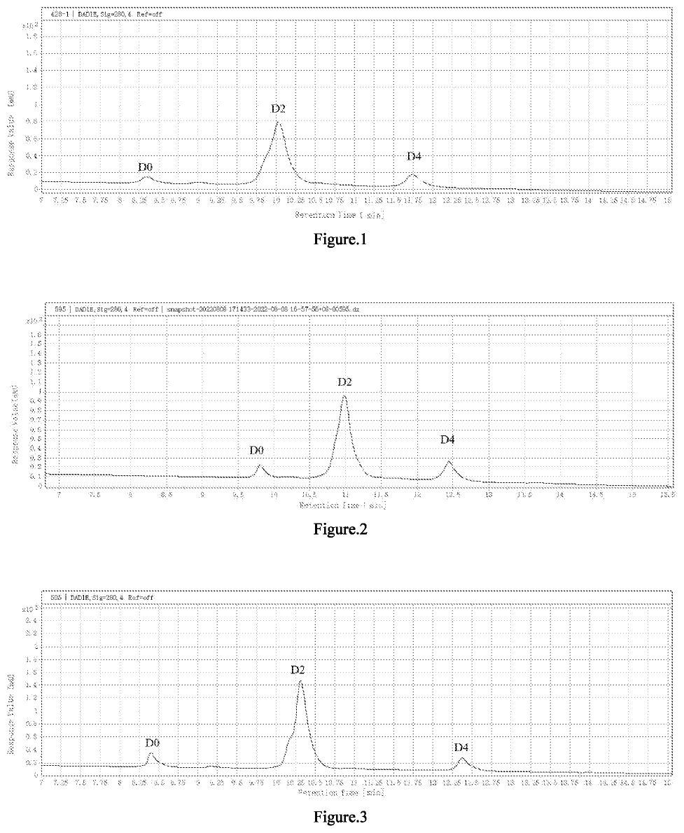

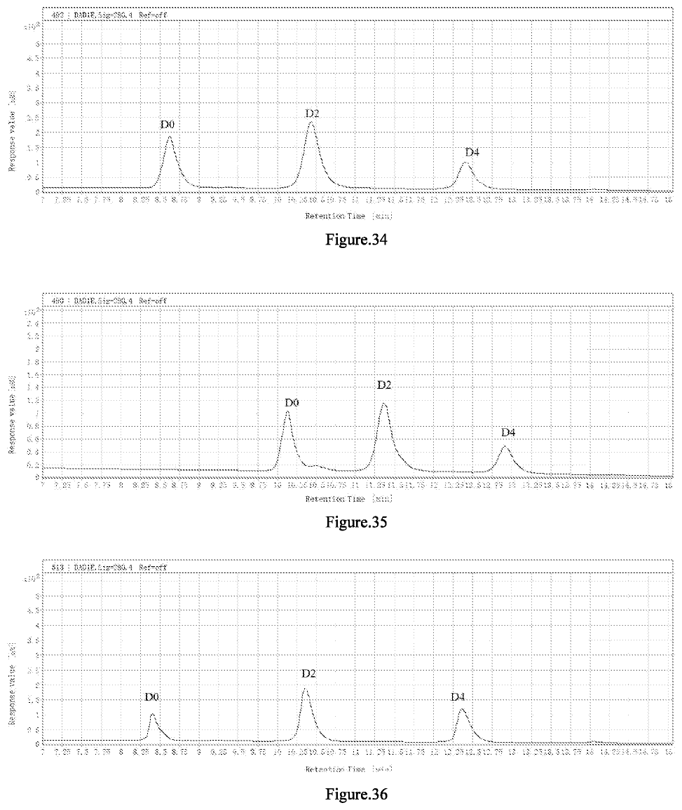

As shown in table 4, FIGS. 1-3 and 34-36, the results demonstrate that the content of D2 in examples 1-3 is generally up to 70%, such as 75%, 80% and 82%. In contrast, the content of D2 in comparative examples 1-3 is generally less than 50%. These results clearly demonstrate that the ADCs prepared with the method of the present application has a significantly improved homogeneity. By using the transitional metal ions and TCEP with specific molar ratio, the content of D2 is up to 65%, even to 70%, 75%, 80% or 82%.

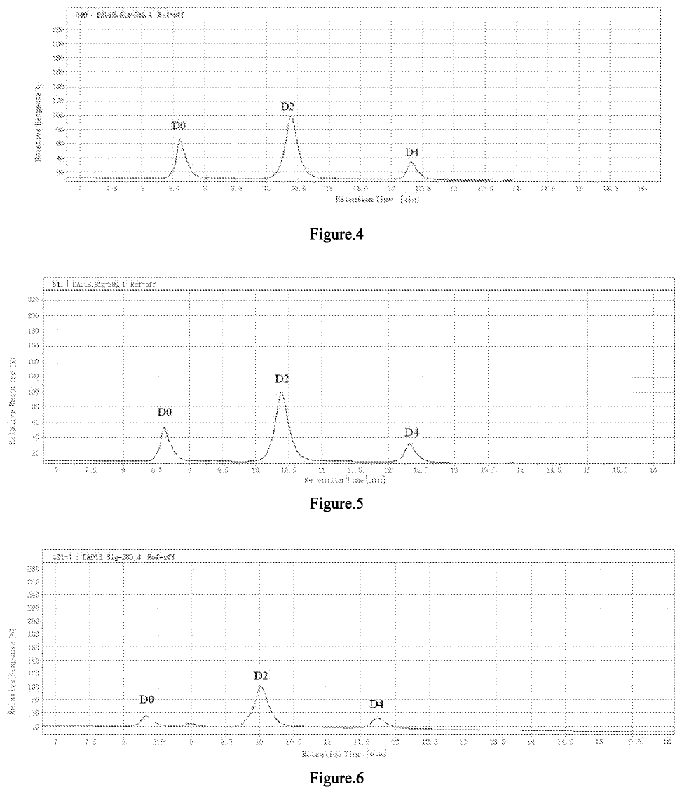

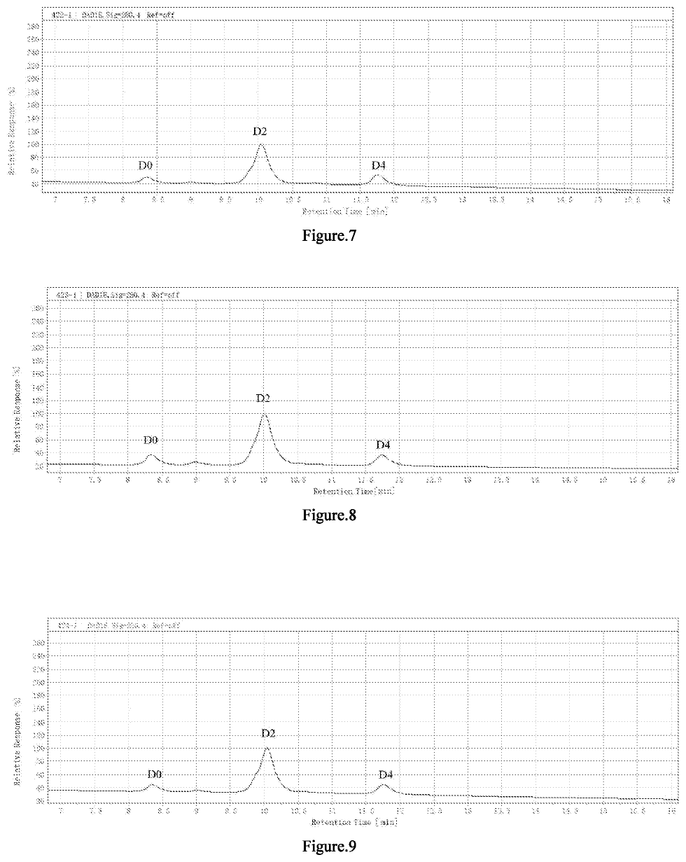

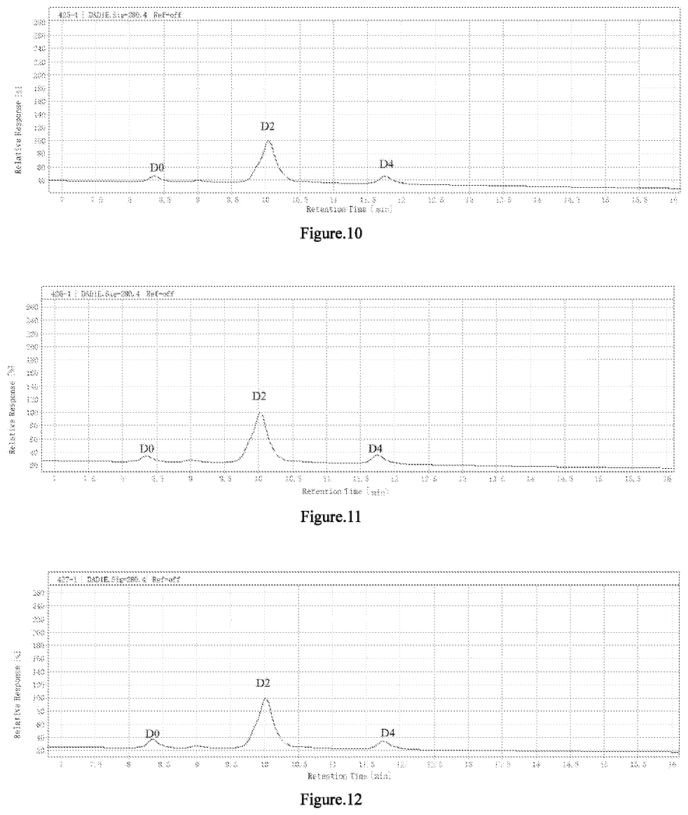

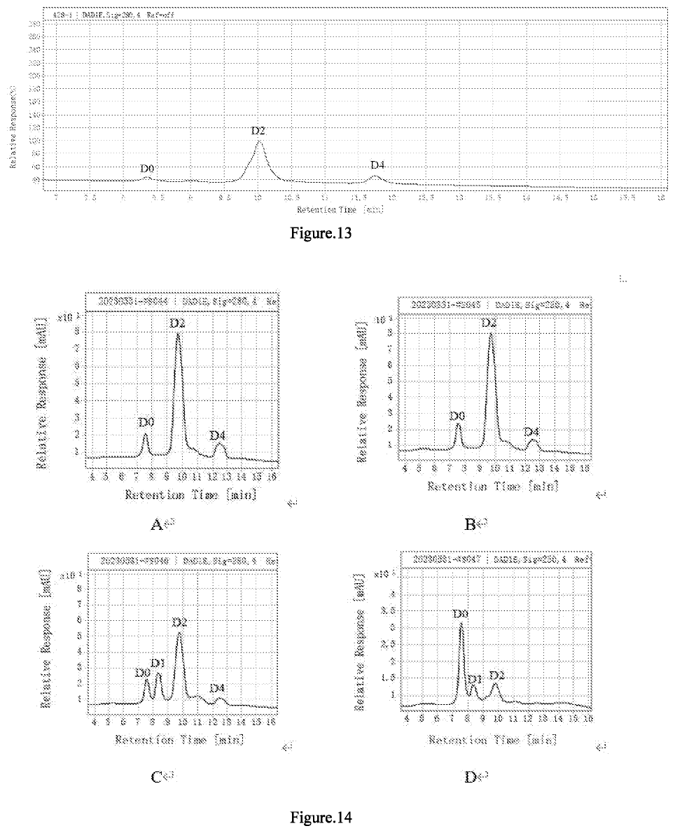

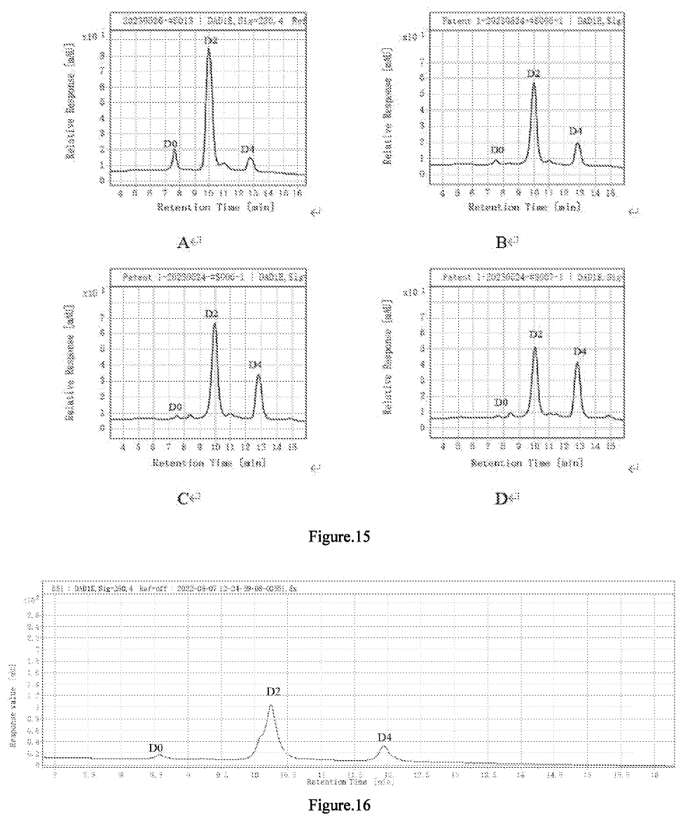

TABLE 5
the results of homogeneity assays of examples 4-16 and comparative example 4

| | | The molar ratio of | | | | | |
|---|---|---|---|---|---|---|---|
| No. | FIG. | Zn²⁺ and TCEP | D0(%) | D1(%) | D2(%) | D4(%) | D6(%) |
| Example 4 | FIG. 4 | 0.4:1 | 27.12 | 0 | 59.13 | 13.75 | 0 |
| Example 5 | FIG. 5 | 1:1 | 21.73 | 0 | 64.68 | 13.59 | 0 |
| Example 6 | FIG. 6 | 2:1 | 12.63 | 0 | 71.36 | 16.01 | 0 |
| Example 7 | FIG. 7 | 4:1 | 7.49 | 0 | 75.48 | 17.02 | 0 |
| Example 8 | FIG. 8 | 6:1 | 8.28 | 0 | 77.93 | 13.79 | 0 |
| Example 9 | FIG. 9 | 8:1 | 8.28 | 0 | 77.39 | 14.34 | 0 |
| Example 10 | FIG. 10 | 10:1 | 7.21 | 0 | 79.13 | 13.66 | 0 |
| Example 11 | FIG. 11 | 12:1 | 6.41 | 0 | 81.04 | 12.55 | 0 |
| Example 12 | FIG. 12 | 14:1 | 8.97 | 0 | 80.75 | 10.28 | 0 |
| Example 13 | FIG. 13 | 16:1 | 5.44 | 0 | 80.75 | 13.81 | 0 |
| Example 14 | FIG. 14 A | 30:1 | 8.55 | 0 | 81.60 | 9.85 | 0 |
| Example 15 | FIG. 14 B | 70:1 | 10.16 | 0 | 82.26 | 7.59 | 0 |
| Example 16 | FIG. 14 C | 125:1 | 10.55 | 15.34 | 68.47 | 5.65 | 0 |
| Comparative | FIG. 14 D | 250:1 | 64.17 | 14.17 | 21.66 | 0 | 0 |
| Example 4 | | | | | | | |

As show in table 5, FIGS. 4-14, the results showed the content of D2 increases as the molar ratio of Zn²⁺ and TCEP increases from 0.4:1 to 12:1. After that, the content of D2 ratio reaches a plateau. when the molar ratio of Zn²⁺ and TCEP is 250:1, the content of D2 is lower than that of the molar ratio of the Zn²⁺/TCEP-NO ranging from 0.4:1 to 125:1. Those results indicated that the molar ratio of Zn²⁺ and TCEP plays a key role in determining the content of D2 and the selective reduction. When the molar ratio of Zn²⁺ and TCEP is 0.4:1 to 200:1, the content of D2 is up to 60%, 65%, even to 70%, 75%, 80% or 83%.

TABLE 6
the results of homogeneity assays of examples 17-20

| | | Antibody/TCEP | D0 | D2 | D4 | D6 |
|---|---|---|---|---|---|---|
| No. | FIG. | (Molar Ratio) | (%) | (%) | (%) | (%) |
| E17 | FIG. 15 A | 1:1.2 | 9.27 | 82.64 | 8.09 | 0 |
| E18 | FIG. 15 B | 1:2.0 | 2.72 | 78.12 | 19.16 | 0 |
| E19 | FIG. 15 C | 1:2.5 | 2.75 | 68.75 | 28.50 | 0 |
| E20 | FIG. 15 D | 1:3.0 | 1.94 | 56.33 | 39.47 | 2.26 |

“E” was short for Example.

The results of Examples 17-20 are shown in Table 6, and the chromatograms are shown in FIG. 15. As the results shown in the table 6, when the molar ratio of antibody/TCEP is 1:1 to 1:3.0, the content of the ADC with D2 is up to 56%, 70%, even to 80%. When the molar ratio of antibody/TCEP is 1:2 and 1:3, the reduction time is shortened to 1 h and the content of D2 is greater than 56%, even to 70% and 78%.

TABLE 7
the results of homogeneity assays of examples 21-34 and comparative examples 5-12

| | The buffer system | The pH | D0(%) | D1(%) | D2(%) | D4(%) | D6(%) |
|---|---|---|---|---|---|---|---|
| Example 21 | MOPS buffer | 6.7 | 2.6 | 0 | 79.47 | 17.93 | 0 |
| Example 22 | MOPS buffer | 7.0 | 3.66 | 0 | 80.03 | 16.3 | 0 |
| Example 23 | MOPS buffer | 7.4 | 1.8 | 0 | 75.37 | 22.84 | 0 |
| Example 24 | Bis-Tris buffer | 6.7 | 6.25 | 0 | 81.57 | 12.18 | 0 |
| Example 25 | PIPES buffer | 6.7 | 8.41 | 0 | 81.08 | 10.51 | 0 |
| Example 26 | BES buffer | 6.7 | 3.47 | 0 | 77.61 | 18.92 | 0 |
| Example 27 | MES buffer | 6.7 | 2.64 | 0 | 76.79 | 20.56 | 0 |
| Example 28 | HEPES buffer | 6.7 | 15.42 | 0 | 77.8 | 6.78 | 0 |
| Example 29 | DIPSO buffer | 7.4 | 10.48 | 0 | 66.33 | 23.19 | 0 |
| Example 30 | MOPSO buffer | 7.4 | 12.08 | 0 | 66.25 | 21.67 | 0 |
| Example 31 | TES buffer | 7.4 | 15.96 | 0 | 64.79 | 19.25 | 0 |
| Example 32 | ACES buffer | 7.4 | 14.81 | 0 | 60.34 | 22.81 | 2.04 |
| Example 33 | MES buffer | 5.8 | 18.45 | 5.69 | 64.47 | 11.39 | 0 |
| Example 34 | MES buffer | 6.4 | 9.45 | 0 | 79.90 | 10.65 | 0 |
| Comparative Example 5 | PB buffer | 5.8 | 76.09 | 0 | 23.91 | 0 | 0 |
| Comparative Example 6 | PB buffer | 6.2 | 77.93 | 0 | 22.07 | 0 | 0 |
| Comparative Example 7 | PB buffer | 6.7 | 80.3 | 0 | 19.7 | 0 | 0 |
| Comparative Example 8 | PB buffer | 7.0 | 75.07 | 0 | 24.93 | 0 | 0 |
| Comparative Example 9 | PB buffer | 7.4 | 62.8 | 0 | 35.85 | 1.34 | 0 |
| Comparative Example 10 | ADA buffer | 6.7 | 40.06 | 0 | 51.99 | 7.95 | 0 |
| Comparative Example 11 | MOBS buffer | 7.4 | 31.01 | 0 | 53.63 | 15.36 | 0 |
| Comparative Example 12 | TAPSO buffer | 7.4 | 25.42 | 0 | 50.24 | 22.07 | 2.27 |

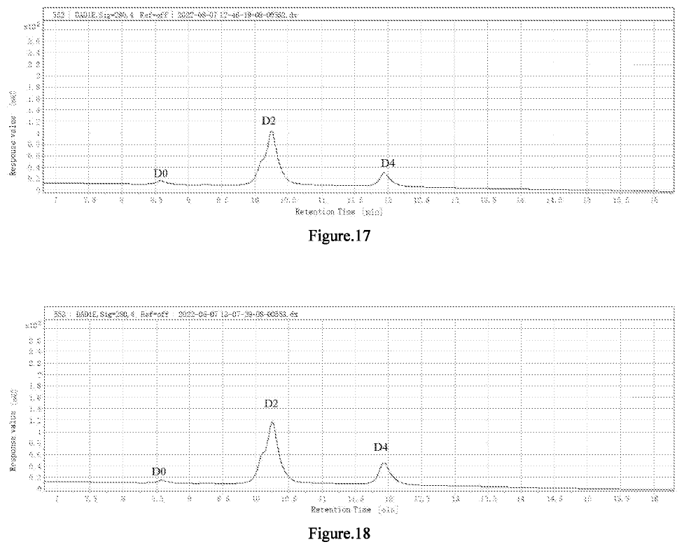

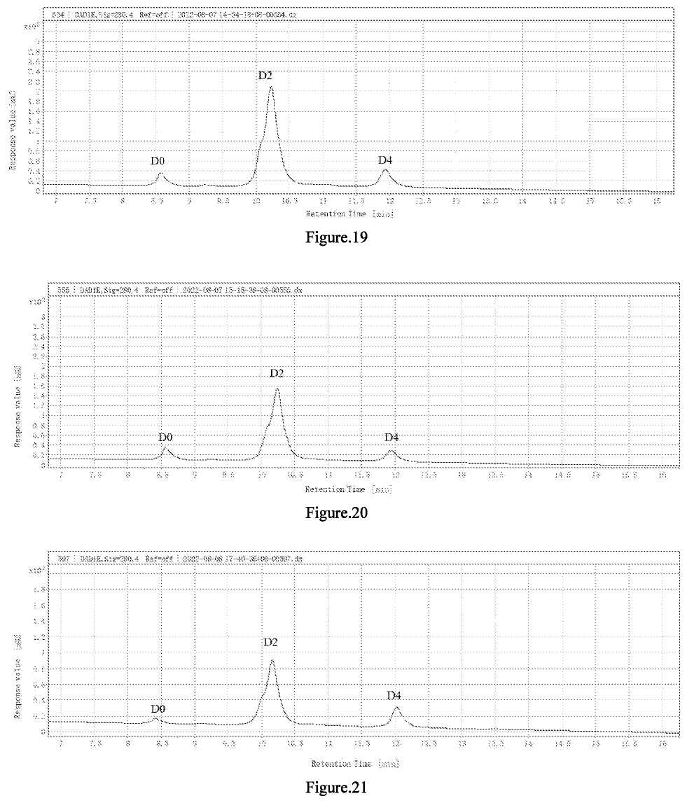

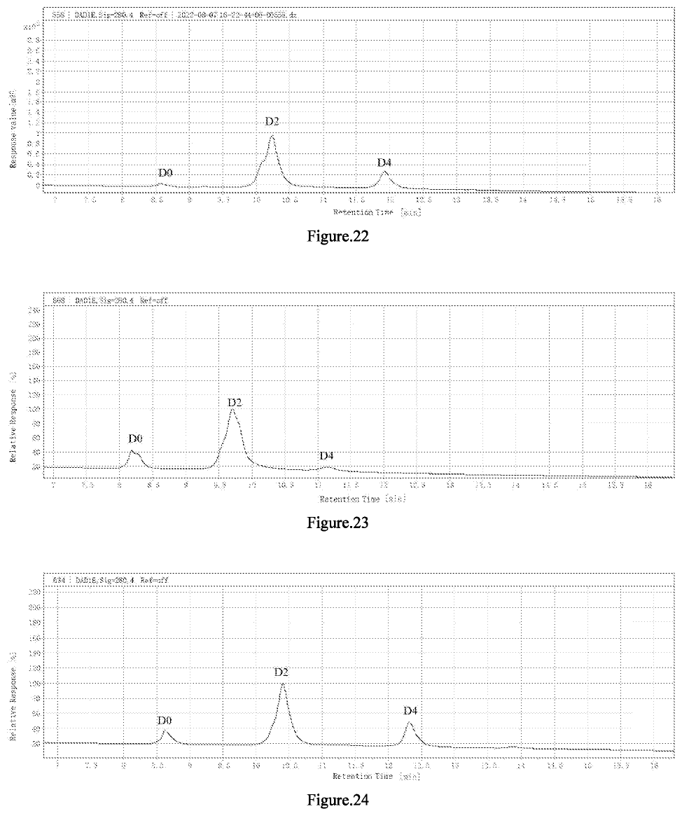

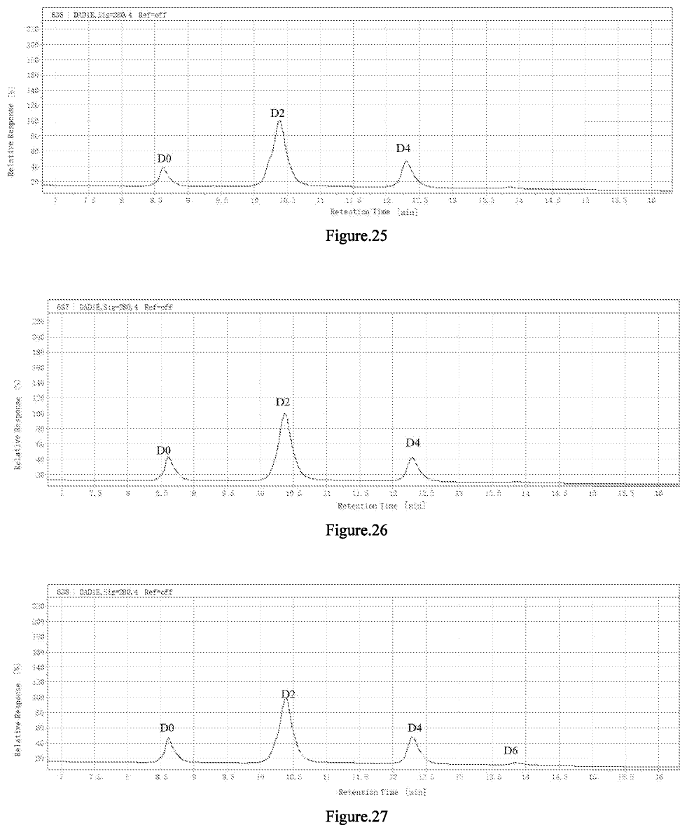

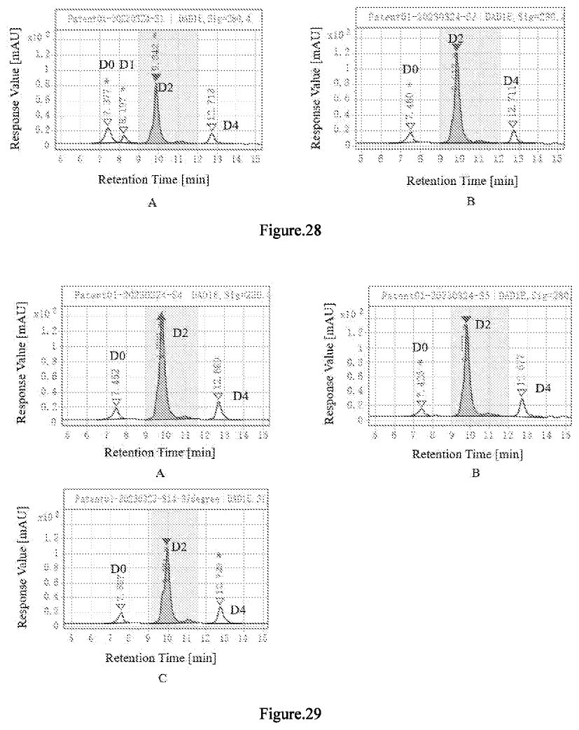

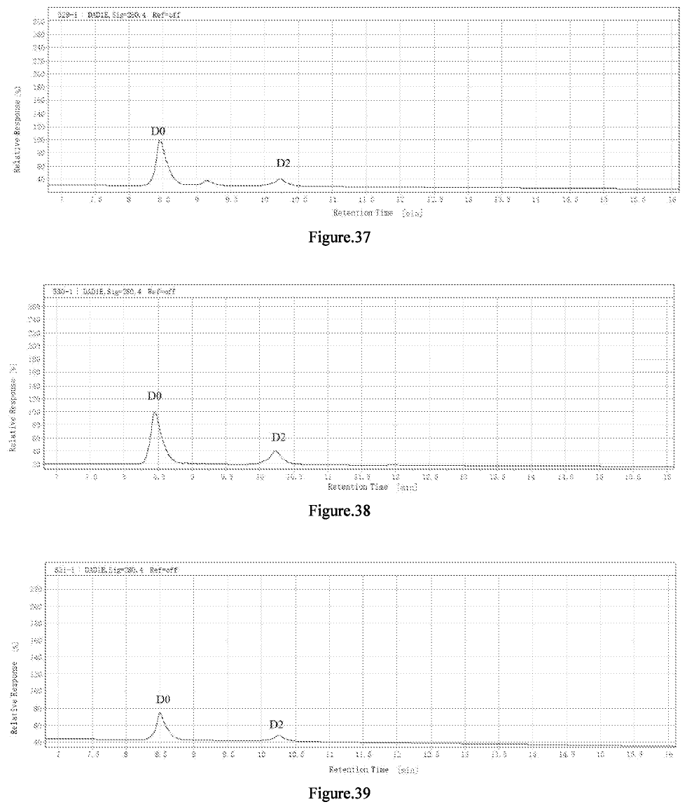

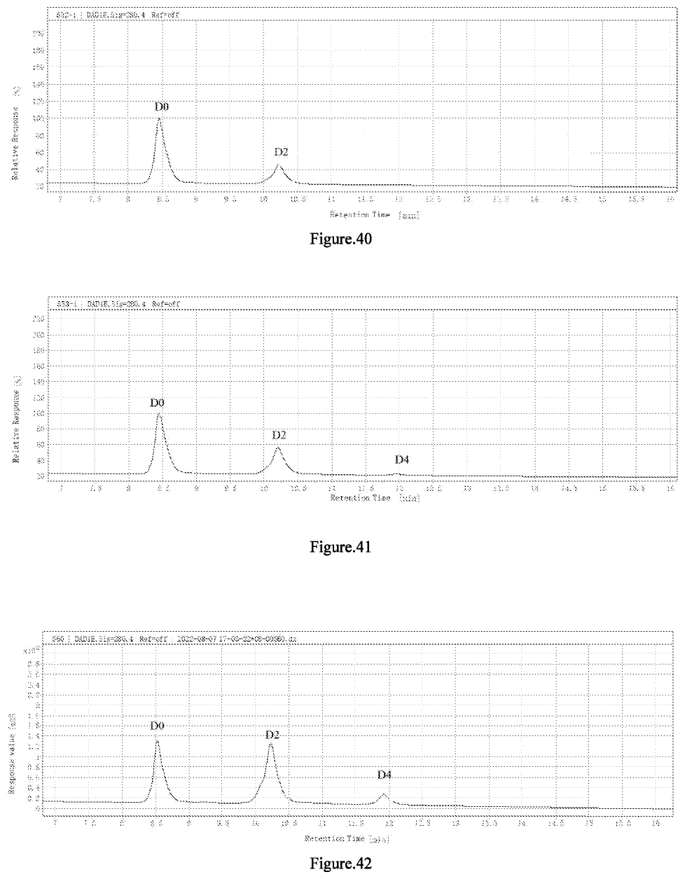

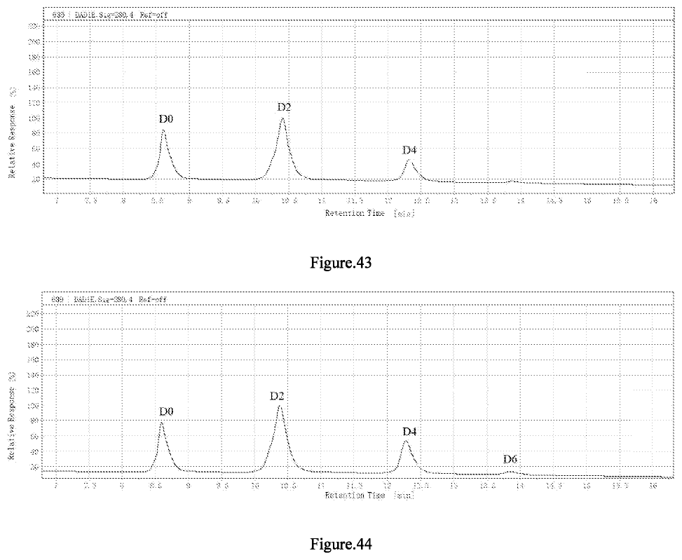

As show in table 7, FIGS. 16-28, 37-44, the results showed the types and the pH value of the buffer system will impact the content of D2. The buffer systems of examples 21-34 are useful to increase the content of D2.

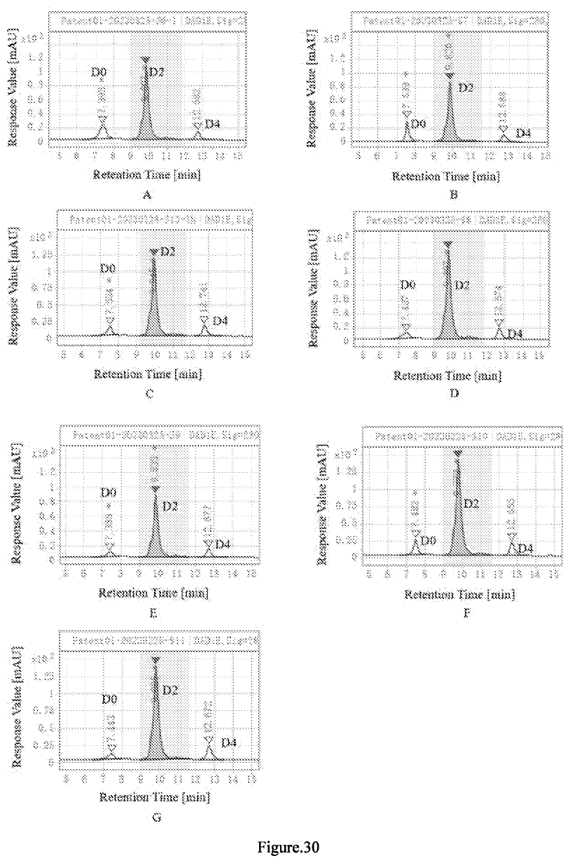

TABLE 8
The results of homogeneity assays of examples 35-44

| | | The reduction temperature | The reduction time | | | | |
|---|---|---|---|---|---|---|---|
| No. | FIG. | in step (1) (° C.) | in step (1) (h) | D0(%) | D2(%) | D4(%) | D6(%) |
| E35 | FIG. 29 A | 15 | 4 | 7.87 | 79.88 | 12.24 | 0 |
| E36 | FIG. 29 B | 24 | 4 | 6.66 | 78.73 | 14.61 | 0 |
| E37 | FIG. 29 C | 37 | 4 | 9.09 | 75.89 | 15.02 | 0 |
| E38 | FIG. 30 A | 4 | 1 | 17.36 | 75.30 | 7.33 | 0 |
| E39 | FIG. 30 B | 4 | 2 | 15.32 | 76.50 | 8.17 | 0 |
| E40 | FIG. 30 C | 4 | 3 | 9.47 | 80.64 | 9.90 | 0 |
| E41 | FIG. 30 D | 4 | 4 | 7.38 | 83.34 | 9.27 | 0 |
| E42 | FIG. 30 E | 4 | 5 | 8.03 | 81.64 | 10.33 | 0 |
| E43 | FIG. 30 F | 4 | 6 | 9.98 | 80.53 | 9.49 | 0 |
| E44 | FIG. 30 G | 4 | 8 | 5.99 | 83.28 | 10.73 | 0 |

As shown in example 1, and examples 35-37, the results showed the content of D2 is up to 75%, even to 80% when the reductant temperature in step (1) is from 4° C. to 37° C.

As shown in examples 38-44, the results showed that the content of D2 is up to 75%, 80%, even to 83% when the reductant time in step (1) is from 1 h to 8 h. The content of D2 increases when the reduction time in step (1) is from 1 h to 4 h, and it reaches a plateau after 4 h.

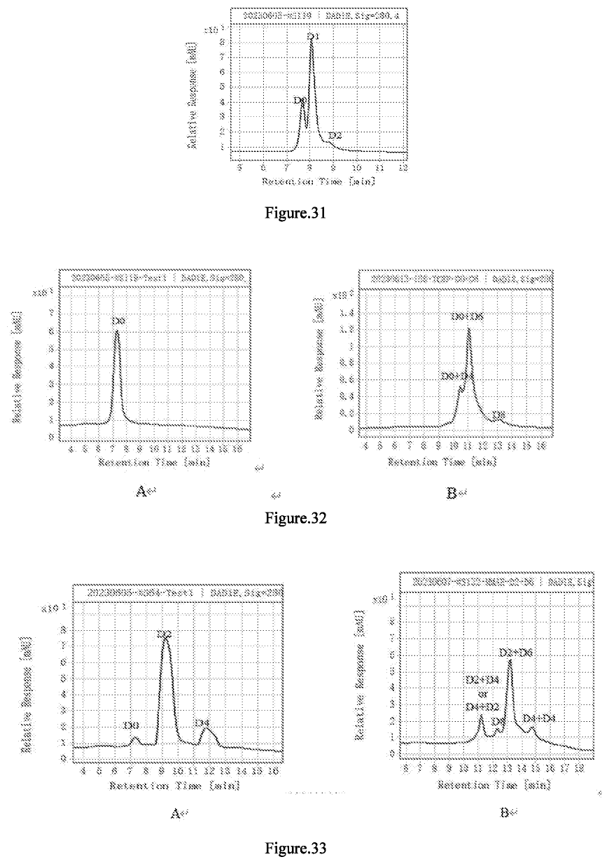

TABLE 9

| | | The type of | D0 | D1 | D2 |
|---|---|---|---|---|---|
| Example | FIG. | the ADCs | (wt %) | (wt %) | (wt %) |
| 45 | FIG. 31 | D1 | 20.62 | 77.71 | 1.67 |

TABLE 10

| No. | FIG. | D0(%) | / | / |
|---|---|---|---|---|
| E46-step (2) | FIG. 32A | 100 | / | / |

| | | D0 + | D0 + | |
|---|---|---|---|---|
| No. | FIG. | D4 (%) | D6 (%) | D8 (%) |
| E46-step (4) | FIG. 32B | 19.33 | 73.50 | 7.17 |

TABLE 11

| No. | FIG. | D0(%) | D2 (%) | D4 (%) | / |
|---|---|---|---|---|---|
| E47-step (2) | FIG. 33A | 3.49 | 83.29 | 13.22 | / |

| | | D2 + D4/ | | D2 + | D4 + |
|---|---|---|---|---|---|
| No. | FIG. | D4 + D2(%) | D8 (%) | D6 (%) | D4 (%) |
| E47-step (4) | FIG. 33B | 16.91 | 3.19 | 72.43 | 7.47 |

As shown in table 9, and FIG. 31, the results demonstrate that the content of the ADC with D1 is generally up to 77.71%. As shown in table 10, and FIG. 32, the results demonstrate that the content of the ADC with D0+D6 is generally up to 73.50%. As shown in table 11, and FIG. 33, the results demonstrate that the content of the ADC with D2+D6 is generally up to 72.43%. Those results indicate the method of the present application could modify the antibody with site-specific and prepare the different kinds of ADCs with improving the homogeneity.

To sum up, the method of the present application can increase the homogeneity of ADCs. Specifically, the ADCs prepared by using the transitional metal ions and TCEP with specific molar ratio, contain D2 in general more than 55%, 60%, 65%, even to more than 70%, 75% and 80%. Meanwhile, the method of the present application is simple to operate without antibody engineering and enzymes, and it is fully compatible with current thiol-reactive linker-drug technologies.

1.-46. (canceled)

47. A method of preparing an antibody with thiol group site-specific modifications, which characterized in that, the thiol group(s) is/are reduced from the interchain disulfide bonds within the antibody, and the method comprises using tris (2-carboxyethyl) phosphine (TCEP) or a salt thereof and transition metal ions, wherein, the molar ratio of TCEP and the transition metal ions is 1:0.4 to 1:200.

48. The method according to claim 47, which characterized in that, the number of the thiol group(s) is/are 1, 2, 3, 4, 5, 6, 7 or 8.

49. The method according to claim 48, which characterized in that, the interchain disulfide bonds connect the two upper heavy chains in the hinge region, and/or the heavy chain to the light chain in the Fab region.

50. The method according to claim 47, which characterized in that, the method comprises the following steps,
(a) incubating TCEP or the salt thereof and the antibody in the presence of the transition metal ions in a first buffer system to selectively reduce the interchain disulfide bonds within the antibody, which allows a bearing of two reduced thiol groups of the antibody, and the molar ratio of TCEP and the transition metal ions is 1:0.4 to 1:70;
(b) introducing metal chelators and a modification reagent 1 to react with the reduced thiol groups resulted from step (a) in the first buffer system, wherein, the modification reagent 1 is an end capping reagent, a first linker-payload or a first thiobridge reagent.

51. The method according to claim 50, which characterized in that, the method further comprises the following steps,
(c) incubating the reaction product from step (b) and a second reductant in a second buffer system to reduce the interchain disulfide bonds in the reaction product;
(d) introducing the incubation product from step (c) and a modification reagent 2 to react with the reduced thiol groups resulted from step (c), wherein, the modification reagent 2 is a second linker-payload or a second thiobridge reagent.

52. The method according to claim 51, which characterized in that, the method further comprises the following steps,
(c) incubating the reaction product from step (b) and the second reductant in the second buffer system to reduce the interchain disulfide bonds in the reaction product, introducing the transition metal ions;
(d) introducing the incubation product from step (c) and the modification reagent 2 to react with the reduced thiol groups resulted from step (c), introducing the metal chelators.

53. The method according to claim 51, which characterized in that, the first thiobridge reagent bears the first linker-payload or reactive groups, and/or the second thiobridge reagent bears the second linker-payload or the reactive groups.

54. The method according to claim 51, which characterized in that, the first thiobridge reagent and the second thiobridge reagent independently contain at least two substituted groups allowing a re-bridging of the thiol groups.

55. The method according to claim 54, which characterized in that, the first thiobridge reagent and the second thiobridge reagent are independently selected from the group consisting of

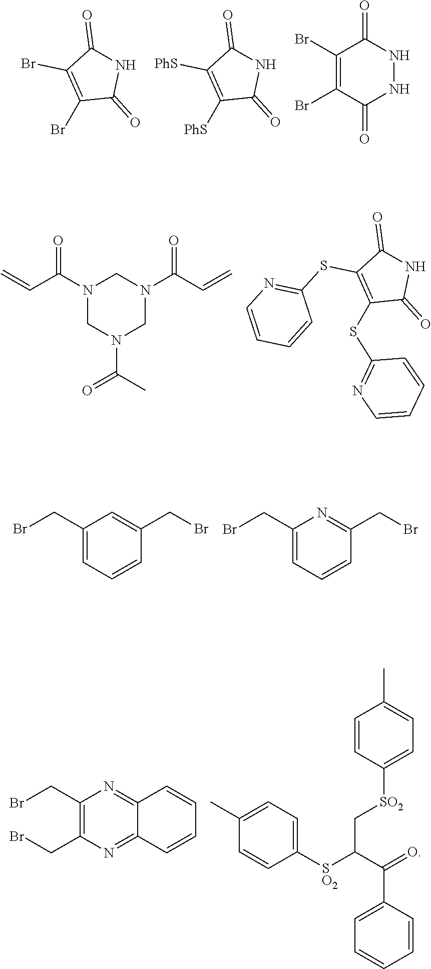

56. The method according to claim 54, which characterized in that, the reactive groups independently contain azido and/or dibenzocyclooctyne (DBCO).

57. The method according to claim 50, which characterized in that, the molar ratio of TCEP and the transition metal ions is 1:2 to 1:70.

58. The method according to claim 50, which characterized in that, the molar ratio of TCEP and the metal transition ions is 1:12.

59. The method according to claim 50, which characterized in that, the molar ratio of the antibody and TCEP is 1:1 to 1:3.

60. The method according to claim 59, which characterized in that, the molar ratio of the antibody and TCEP is 1:1 to 1:2.

61. The method according to claim 51, which characterized in that, the first buffer system and the second buffer system are independently selected from a group consisting of HEPES buffer, Histidine buffer, PBS, MES buffer, BES buffer, MOPS buffer, Bis-Tris buffer, Acetate buffer, DIPSO buffer, MOPSO buffer, TES buffer, ACES buffer, TAPSO buffer, PIPES buffer, BTP buffer, HEPPSO buffer, POPSO buffer, EPPS buffer and Tris buffer.

62. The method according to claim 61, which characterized in that, the first buffer system and the second buffer system are independently selected from a group consisting of Bis-Tris buffer, MOPS buffer, MES buffer, HEPES buffer, PIPES buffer, DIPSO buffer, MOPSO buffer, TES buffer and BES buffer.

63. The method according to claim 51, which characterized in that, the pH value of the first buffer system and the second buffer system is 5.5 to 8.

64. The method according to claim 63, which characterized in that, the pH value of the first buffer system and the second buffer system is 6.7 to 7.4.

65. The method according to claim 47, which characterized in that, said transition metal ions are selected from a group consisting of Zn²⁺, Cd²⁺, Hg²⁺, Ni²⁺, Co²⁺ and the combination thereof.

66. The method according to claim 50, which characterized in that, the incubation temperature is 0° C. to 37° C. in step (a), the incubation time is 0.5 h to 24 h in step (a).

67. The method according to claim 66, which characterized in that, the incubation temperature is 4° C. to 24° C. in step (a), and the incubation time is 2 h to 16 h in step (a).

68. The method according to claim 50, which characterized in that, the molar ratio of the antibody and TCEP is 1:3, and the incubation time is 1 h to 5 h.

69. The method according to claim 51, in step (c), the molar ratio of the second reductant and the transition metal ions is 1:0.05 to 1:40, and/or the molar ratio of the antibody and the second reductant is 1:2.5 to 1:20, and/or the incubation time is 1 h to 24 h.

70. The method according to claim 51, in step (c), the molar ratio of the second reductant and the transition metal ions is 1:0.4 to 1:100, and/or the molar ratio of the antibody and the second reductant is 1:0.8 to 1:2.5, and/or the incubation time is 0.5 h to 24 h.

71. The method according to claim 51, which characterized in that, when the first thiobridge reagent bears the reactive groups, the step (b) comprises the following step:
introducing the metal chelators and the first thiobridge reagent bearing reactive groups to re-bridge the reduced thiol groups resulted from step (a), then, incubating the first linker-payload in the first buffer system to react with the reactive groups of the thiobridge group; and/or
when the second thiobridge reagent bears the reactive groups, the step (d) comprises the following step:
introducing the incubation product from step (c) and the second thiobridge reagent bearing the reactive groups to re-bridge the reduced thiol groups resulted from step (c), then, incubating the second linker-payload in the second buffer system to react with the reactive groups of the thiobridge group.

72. The method according to claim 52, which characterized in that, when the second thiobridge reagent bears the reactive groups, the step (d) comprises the following step:
introducing the incubation product from step (c) and the second thiobridge reagent bearing the reactive groups to re-bridge the reduced thiol groups resulted from step (c), introducing the metal chelators, then, incubating the second linker-payload in the second buffer system to react with the reactive groups of the thiobridge group.

73. The method according to claim 47, which characterized in that, said method comprising the following steps,
(a1) incubating TCEP or the salt thereof and the antibody in the presence of an effective amount of the transition metal ions in the first buffer system to selectively reduce one of four interchain disulfide bonds within the antibody, the molar ratio of TCEP and said transition metal ions is 1:1 to 1:70;
(b1) introducing an excess amount of the metal chelators and an excess amount of the first linker-payload to react with the reduced thiol groups resulted from step (a1).

74. The method according to claim 73, which characterized in that, the molar ratio of TCEP and said transition metal ions is 1:2 to 1:16.

75. The method according to claim 73, which characterized in that, the molar ratio of TCEP and said transition metal ions is 1:12.

76. The method according to claim 73, which characterized in that, the antibody with thiol group site-specific modifications is an antibody drug conjugate (ADC) with D2.

77. The method according to claim 73, which characterized in that, said method comprising the following steps,
(c2) incubating the reaction product from (b1) and the second reductant in the second buffer system to reduce the interchain disulfide bonds within the reaction product from (b1);
(d2) introducing the incubation product from step (c2) and an excess amount of the second linker-payload to react with the reduced thiol groups resulted from step (c2).

78. The method according to claim 77, which characterized in that, the antibody with thiol group site-specific modifications is the ADC with D2+D6.

79. The method according to claim 51, which characterized in that, said method further comprises the following steps,
introducing a compound that contains at least one thiol to consume excessive said first linker-payload in step (b) and/or said second linker-payload in step (d);
purifying and recovering the resultant antibody with thiol group site-specific modifications in step (b) and/or in step (d).

80. The method according to claim 47, which characterized in that, the antibody is a monoclonal antibody, a polyclonal antibody, a mono-specific antibody or a multi-specific antibody.

81. The method according to claim 80, which characterized in that, the antibody is Transtuzumab, Sacituzumab or Belantamab.

82. The method according to claim 51, which characterized in that, a linker of the first linker-payload and the second linker-payload is selected from any one of which the one terminal can be connected to the reduced thiol group of the antibody or the reactive groups of the thiobridge reagent, and the other terminal can be connected to the payload.

83. The method according to claim 73, which characterized in that, the linker is maleimidocaproyl-valine-citrulline-p-aminobenzoyloxycarbonyl (MC-VC-PAB).

84. The method according to claim 51, which characterized in that, the payload is selected from any one of which contains at least one substituted group allowing a connection from the payload to the linker.

85. The method according to claim 73, which characterized in that, the payload is monomethyl auristatin E (MMAE)

86. An antibody with thiol group site-specific modifications prepared by the method of claim 47.

87. The antibody with thiol group site-specific modifications according to claim 86, which characterized in that, the antibody with thiol group site-specific modifications is the ADC with D2, the ADC with D1, the ADC with D2+D6, the ADC with D2+D3, the ADC with D1+D6, the ADC with D1+D3, the ADC with D0+D6 or the ADC with D0+D3.

88. A pharmaceutical composition comprising the antibody with thiol group site-specific modifications according to claim 86 and one or more of pharmaceutically acceptable carrier.

89. A method of preventing or treating a disease in a subject in need thereof, comprising administrating to the subject a therapeutically effective amount of the antibody with thiol group site-specific modifications according to claim 86.
